(19) United States
(12) Patent Application Publication
(10) Pub. No.: US 2022/0378570 A1
(43) Pub. Date: Dec. 1, 2022

(54) VASCULAR CASTING AND APPLICATIONS THEREOF

(71) Applicant: 3D Systems, Inc., Houston, TX (US)

(72) Inventors: Bagrat GRIGORYAN, Houston, TX (US); Jordan MILLER, Houston, TX (US)

(21) Appl. No.: 17/751,029

(22) Filed: May 23, 2022

Related U.S. Application Data
(60) Provisional application No. 63/192,932, filed on May 25, 2021.

(51) Int. Cl. A61F 2/06; A61L 27/50; A61L 27/28; B29C 39/12; B29C 39/38; B22D 19/00; B33Y 80/00

(52) U.S. Cl. CPC A61F 2/06; A61L 27/507; A61L 27/28; B29C 39/123; B29C 39/38; B22D 19/00; B33Y 80/00; A61F 2240/004; A61L 2420/02; A61L 2420/08; B29K 2705/00

(57) ABSTRACT

A method of preparing a structure is provided. The method includes providing an initial structure; casting a first material in one or more void volumes of the initial structure; removing the initial structure from the first material; obtaining a cast structure comprising the first material; coating a second material on the cast structure; casting a third material using the coated cast structure; removing the first material; and obtaining a final structure. In various embodiments, the initial structure can include a first initial structure and a second initial structure and casting a first material in one or more first void volumes of the first initial structure and in one or more second void volumes of the second initial structure. In various embodiments, the method includes assembling the first cast structure and the second cast structure and obtaining an assembled structure comprising the first cast structure and the second cast structure.

### CROSS-REFERENCE TO RELATED APPLICATIONS

The present application claims the benefit of U.S. Provisional Application No. 63/192,932, filed on May 25, 2021, the contents of which are incorporated herein by reference as if set forth in full.

### BACKGROUND

Producing minute features at the micro- or nanoscale is challenging. Currently available processes and techniques are limited, at least in one way, by their serial approaches to producing such small features, which are not suitable for mass-production. Although lithography can be used to produce micro- or nanoscale features, they are often limited to two-dimensional features, which can be post processed to form three-dimensional features. Even more challenging is production of such small features for biological or biocompatible applications. Therefore, there is a need for new and novel approaches suitable for biological or biocompatible applications that can be used in mass-production that can further the development three-dimensional micro- or nanoscale features.

### SUMMARY

In accordance with various embodiments, a method of preparing a structure is provided. The method includes providing an initial structure having one or more features; performing a post-process of the initial structure; casting a material using the post-processed initial structure; removing the initial structure from the cast material; and obtaining a final structure comprising the cast material.

In accordance with various embodiments, a method of preparing a structure is provided. The method includes providing an initial structure; casting a first material in one or more void volumes of the initial structure; removing the initial structure from the first material; obtaining a cast structure comprising the first material; casting a second material using the cast structure; removing the cast structure from the second material; and obtaining a final structure comprising the second material.

In accordance with various embodiments, a method of preparing a structure is provided. The method includes providing an initial structure; casting a first material in one or more void volumes of the initial structure; removing the initial structure from the first material; obtaining a cast structure comprising the first material; coating a second material on the cast structure; removing the first material from the coated second material; and obtaining a final structure comprising the coated second material.

In accordance with various embodiments, a method of preparing a structure is provided. The method includes providing an initial structure; casting a first material in one or more void volumes of the initial structure; removing the initial structure from the first material; obtaining a cast structure comprising the first material; coating a second material on the cast structure; casting a third material using the coated cast structure; removing the first material; and obtaining a final structure.

In accordance with various embodiments, a method of preparing a structure is provided. The method includes providing a first initial structure and a second initial structure; casting a first material in one or more first void volumes of the first initial structure and in one or more second void volumes of the second initial structure; removing the first initial structure and the second initial structure; obtaining a first cast structure and a second cast structure each comprising the first material; assembling the first cast structure and the second cast structure; obtaining an assembled structure comprising the first cast structure and the second cast structure; casting a third material using the assembled structure; removing the first material; and obtaining a final structure.

In accordance with various embodiments, a structure produced according to any of preceding methods is provided.

In accordance with various embodiments, a system for producing a structure according to any of preceding methods is provided. The system includes an initial structure having a hydrogel, a chamber to house the hydrogel, a manifold to connect to hydrogel vessel inlets and outlets, one or more tubing connected to the manifold, one or more pumps, one or more fluid reservoirs, and/or one or more waste containers.

These and other aspects and implementations are discussed in detail below. The foregoing information and the following detailed description include illustrative examples of various aspects and implementations and provide an overview or framework for understanding the nature and character of the claimed aspects and implementations. The drawings provide illustration and a further understanding of the various aspects and implementations and are incorporated in and constitute a part of this specification.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 is a flowchart for an example method for preparing a structure, according to various embodiments;

FIG. 2 is a flowchart for an example method for preparing a structure, according to various embodiments;

FIG. 3 is a flowchart for an example method for preparing a structure, according to various embodiments;

FIG. 4 is a flowchart for an example method for preparing a structure, according to various embodiments;

FIG. 5 is a flowchart for an example method for preparing a structure, according to various embodiments; and

FIG. 6 is a block diagram that illustrates a process of transforming an initial structure to a final structure, according to various embodiments.

FIGS. 7A, 7B, and 7C show sample structures prepared using wax, in accordance with various embodiments.

FIGS. 8A and 8B show 3D printed hydrogels composed of polyethylene glycol diacrylate (PEGDA, in yellow) which contained an empty vascular network inside, in accordance with various embodiments.

FIGS. 9A and 9B show example structures comprising a 3D printed PEG hydrogel injected with different dyes, in accordance with various embodiments.

FIGS. 10A and 10B show example structures comprising a 3D printed resin, in accordance with various embodiments.

FIGS. 11A, 11B, and 11C show example released cast structures, in accordance with various embodiments.

FIGS. 12A and 12B show a printed hydrogel structure and a cast structure, respectively, in accordance with various embodiments.

FIGS. 13A and 13B show cast vascular structures, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

It is to be understood that the following disclosure provides many different embodiments, or examples, for implementing different features of the disclosure. Specific embodiments or examples of components and arrangements are described below to simplify the present disclosure. These are, of course, merely examples and are not intended to be limiting. For example, dimensions of elements are not limited to the disclosed range or values but may depend upon process conditions and/or desired properties of the device. Moreover, the formation of a first feature over or on a second feature in the description that follows may include embodiments in which the first and second features are formed in direct contact and may also include embodiments in which additional features may be formed interposing the first and second features, such that the first and second features may not be in direct contact. Various features may be arbitrarily drawn in different scales for simplicity and clarity. Some of the terms used herein are defined as described in this section. Other terms are defined or exemplified elsewhere in the disclosure. Unless defined otherwise, technical and scientific terms used herein have the same meaning as commonly understood by one of ordinary skill in the art to which this invention belongs.

In accordance with various embodiments, various methods and processes for preparing a structure are described herein. In accordance with various embodiments, the disclosed methods, processes, and implementations offer mass-production capable manufacturing techniques for preparing a structure for biological or biocompatible applications. For example, the structure produced the disclosed methods, processes, or implementations can be used for cell culturing processes for distilling drug candidates. In accordance with various embodiments, the (prepared) structure can contain features that mimic human anatomy and physiology and can be used as biomimetic human tissue models for drug discovery or therapeutic applications. In accordance with various embodiments, the prepared structure can comprise cell-adhesive and cell-degradable materials. In accordance with various embodiments, the prepared structure can include cell-adhesive and cell-degradable materials where cells can adhere, grow, and migrate onto a matrix of the prepared structure.

In accordance with various embodiments, the structure can contain one or more features. In accordance with various embodiments, the structure can contain one or more positive or negative features. In accordance with various embodiments, the one or more positive features refer to spaces that are occupied by a material that makes up the structure. In accordance with various embodiments, the one or more negative features refer to spaces that are absent of a material that made up the structure, e.g., a void space or a void volume within the structure. In accordance with various embodiments, the one or more negative features refer to one or more void volumes of the structure.

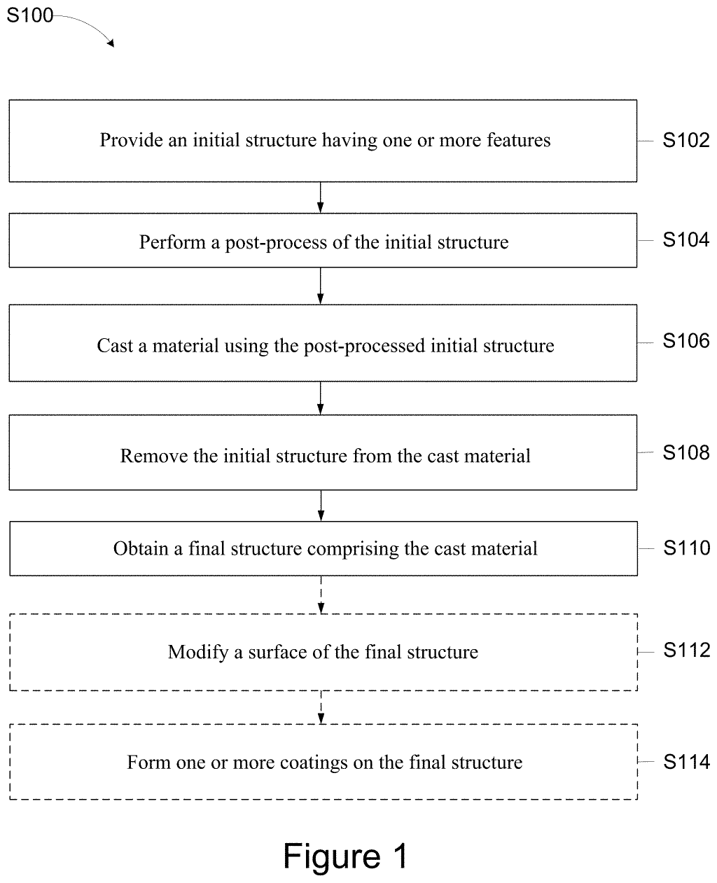

In accordance with various embodiments, the structure, either or both initial structure or final structure, can contain a vascular component (also referred to herein as a vascular topology, one or more features, or one or more void volumes), which enables cells to be under perfusion conditions. In accordance with various embodiments, more than one vascular component (e.g., one or more first features and one or more second features, or one or more final first features and one or more final second features as described with respect to FIGS. 1-5) or more than one vascular topology can be incorporated into the same structure. In accordance with various embodiments, fluids including gases and liquids, such as media, such as bile, urine, air, or blood, can be introduced into the vascular components or vascular topologies. In accordance with various embodiments, a vascular component can be also defined as a bounded void volume topology that is suitable for flow of fluids including liquids and gases. In accordance with various embodiments, a vascular topology includes 3D features relating to or comprising a vessel or one or more networks of vessels, which can be configured to facilitate or transport media, such as, but not limited to, blood, bile, urine, or air.

Various configurations, embodiments, and implementations of the technologies, processes, and methods for preparing a structure are described in further detail with respect to FIGS. 1-6. In accordance with various embodiments, various configurations, embodiments, and implementations of the technologies, processes, and methods disclosed herein for preparing a structure can be applicable to any of the example embodiments and configurations described and presented with respect to the following FIGS. 1-6.

Referring now to the figures, FIG. 1 is a flowchart for a method S100 for preparing a structure, according to various embodiments. In accordance with various embodiments, the method S100 includes, at step S102, providing an initial structure having one or more features. In various embodiments, the one or more features can be positive or negative features. In accordance with various embodiments, the positive features refer to spaces that are occupied by a material that made up the structure. In accordance with various embodiments, the negative features refer to spaces that are absent of a material that made up the structure, e.g., a void space or a void volume within the structure. In accordance with various embodiments, the one or more negative features refer to one or more void volumes of the structure.

In various embodiments, the initial structure is a 3-D hydrogel structure that is made of a hydrogel matrix. In various embodiments, the initial structure can include a wax, a plastic, or polyvinyl alcohol, polyethylene glycol diacrylate (PEGDA) having 250-35,000 Da, PEG-norbornene, MMP-sensitive PEGs (PEG-MMP), gelatin methacrylate, or any combination thereof.

In various embodiments, the initial structure can include a wax from a diverse class of organic compounds. In some embodiments, the wax can be lipophilic and/or malleable solids near ambient temperatures. In various embodiments, the wax can include higher alkanes and lipids, with melting points above about 40° C. (104° F.), melting to give low viscosity liquids. In various embodiments, the wax can be insoluble in water but soluble in organic, nonpolar solvents.

In various embodiments, the wax can be an alkane hydrocarbon, where the alkane hydrocarbon has a formula CH₃(CH₂)ₙCH₃, where n=14-48. In various embodiments, the alkane hydrocarbon can be hexadecane, heptadecane, octadecane, eicosane, heneicosane, docosane, tetracosane, nonacosane, triacontane, hentriacontane, dotriacontane, hexatriacontane, tetracontane, tetratetracontane, or pentacontane. In various embodiments, the wax can be an alkene hydrocarbon, where the alkene hydrocarbon has a formula CH₃(CH₂)ₙCH═CH₂, where n=14-48. In various embodiments, the alkene hydrocarbon can be 1-octadecene.

In various embodiments, the wax can be an unsaturated hydrocarbon fatty alcohol. In various embodiments, the wax can be a saturated hydrocarbon fatty alcohol, where the saturated hydrocarbon can be decyl alcohol, lauryl alcohol, myristyl alcohol, palmityl alcohol, stearyl alcohol, arachidyl alcohol, behenyl alcohol, lignoceryl alcohol, ceryl alcohol, myricyl alcohol, or any combination thereof.

In various embodiments, the wax can be a lipid that contains saturated fatty acid, where the fatty acid has a formula CH₃(CH₂)ₙCOOH, where n=4-30. In various embodiments, the lipid can be hexanoic acid, nonanoic acid, octanoic acid, decanoic acid, methyl nonadecanoate, lauric acid, methyl arachidate, myristic acid, methyl tricosanoate, methyl behenate, heptadecanoic acid, palmitic acid, stearic acid, or melissic acid or any combination thereof.

In various embodiments, the wax can be a lipid that contains an unsaturated fatty acid, where the lipid is arachidonic acid, linolenic acid, palmitoleic acid, oleic acid, or any combinations thereof. In various embodiments, the wax can be a lipid combination that contains multiple saturated fatty acids or unsaturated fatty acids. In various embodiments, the wax can be a lipid combination that contains a saturated fatty acid or an unsaturated fatty acid.

In various embodiments, the wax is plant derived, animal derived, petroleum derived, or synthetic or any combination thereof.

In various embodiments, the wax can be paraffin, soy wax, beeswax (major component is myricyl palmitate), gulf wax, carnauba wax, candelilla wax, polyethylene wax, microcrystalline wax, or any combination thereof.

In various embodiments, the wax can contain additives, such as, for example but not limited to, stearic acid, glyceryl tristearate, sorbitan tristearate, beeswax, lecithin, or resins, such as, for example but not limited to, dammar gum, mastic gum, copal gum, shellac, to enhance mechanical properties.

In various embodiments, the wax can be diluted in peanut oil, sunflower oil, soybean oil canola oil, olive oil, or rice bran oil or any combination thereof.

In various embodiments, the wax can be prepared by transferring it into a container, heating them, then using them. In various embodiments, the wax can be melted at a higher temperature to use in an injection process at high temperature in an oven. In some embodiments, the wax can be processed at room temperature. In various embodiments, the wax can be combined with another wax to obtain desired physical, mechanical, and/or chemical properties. In some instances, more than one wax is combined to warm or to liquefy before mixing them.

In various embodiments, one or more features comprises a vascular topology. In various embodiments, one or more features is generated by additive or subtractive manufacturing. In various embodiments, the initial structure can be printed via a 3-D printer.

In various embodiments, the initial structure can include a vascular topology within a tissue or organ.

In accordance with various embodiments, the method S100 includes, at step S104, performing a post-process of the initial structure. In various embodiments, the post-process can include, among many others, washing the initial structure in a solvent, equilibrating the initial structure in a solvent, crosslinking the initial structure in a lightbox, washing in a media to remove water, incubating the initial structure in a nitrogen box to remove oxygen, or coating the void space, or void volume with a material to aid in filling of a cast material, or any of the combination thereof.

In accordance with various embodiments, the method S100 includes, at step S106, casting a material using the post-processed initial structure. In various embodiments, casting a material includes casting the material in one or more void volumes of the initial structure. In various embodiments, the cast material can include, but not limited to, a biomaterial comprising silk, collagen, gelatin, fibrin, synthetic peptides, hyaluronic acid, polymers comprising alginate, polyurethane, polycaprolactone (PCL), elastomers, collagen methacrylate, collagen methacrylamide, gelatin methacrylate, gelatin methacrylamide, silk methacrylate, silk methacrylamide, hyaluronic acid methacrylate, hyaluronic acid methacrylamide, pluronic diacrylate, pluronic methacrylamide, chondroitin sulfate methacrylate, chondroitin sulfate methacrylamide, elastin methacrylate, elastin methacrylamide, cellulose acrylate, cellulose methacrylamide, dextran methacrylate, dextran methacrylamide, heparin methacrylate, heparin methacrylamide, N-isopropyl acrylamide (NIPAAm), chitosan methacrylate, chitosan methacrylamide, polyethylene glycol norbornene, polyethylene glycol dithiol, thiolated gelatin, thiolated chitosan, thiolated hyaluronic acid, thiolated silk, PEG based peptide conjugates, decellularized ECM of any tissue/organ, wax, plastic, metal, including gallium or indium, or alloys, including Field's metal, gallium-indium, gallium-tin, and gallium-indium-tin, supercooled liquid metal, or any combination thereof.

In various embodiments, the wax for the cast material can be a wax from a diverse class of organic compounds. In some embodiments, the wax can be lipophilic and/or malleable solids near ambient temperatures. In various embodiments, the wax can include higher alkanes and lipids, with melting points above about 40° C. (104° F.), melting to give low viscosity liquids. In various embodiments, the wax can be insoluble in water but soluble in organic, nonpolar solvents.

In various embodiments, the wax can be an alkane hydrocarbon, where the alkane hydrocarbon has a formula CH₃(CH₂)ₙCH₃, where n=14-48. In various embodiments, the alkane hydrocarbon can be hexadecane, heptadecane, octadecane, eicosane, heneicosane, docosane, tetracosane, nonacosane, triacontane, hentriacontane, dotriacontane, hexatriacontane, tetracontane, tetratetracontane, or pentacontane. In various embodiments, the wax can be an alkene hydrocarbon, where the alkene hydrocarbon has a formula CH₃(CH₂)ₙCH═CH₂, where n=14-48. In various embodiments, the alkene hydrocarbon can be 1-octadecene.

In various embodiments, the wax can be an unsaturated hydrocarbon fatty alcohol. In various embodiments, the wax can be a saturated hydrocarbon fatty alcohol, where the saturated hydrocarbon can be decyl alcohol, lauryl alcohol, myristyl alcohol, palmityl alcohol, stearyl alcohol, arachidyl alcohol, behenyl alcohol, lignoceryl alcohol, ceryl alcohol, myricyl alcohol, or any combination thereof.

In various embodiments, the wax can be a lipid that contains saturated fatty acid, where the fatty acid has a formula CH₃(CH₂)ₙCOOH, where n=4-30. In various embodiments, the lipid can be hexanoic acid, nonanoic acid, octanoic acid, decanoic acid, methyl nonadecanoate, lauric acid, methyl arachidate, myristic acid, methyl tricosanoate, methyl behenate, heptadecanoic acid, palmitic acid, stearic acid, or melissic acid or any combination thereof.

In various embodiments, the wax can be a lipid that contains an unsaturated fatty acid, where the lipid is arachidonic acid, linolenic acid, palmitoleic acid, oleic acid, or any combinations thereof. In various embodiments, the wax can be a lipid combination that contains multiple saturated fatty acids or unsaturated fatty acids. In various embodiments, the wax can be a lipid combination that contains a saturated fatty acid or an unsaturated fatty acid.

In various embodiments, the wax is plant derived, animal derived, petroleum derived, or synthetic or any combination thereof.

In various embodiments, the wax can be paraffin, soy wax, beeswax (major component is myricyl palmitate), gulf wax, carnauba wax, candelilla wax, polyethylene wax, microcrystalline wax, or any combination thereof.

In various embodiments, the wax can contain additives, such as, for example but not limited to, stearic acid, glyceryl tristearate, sorbitan tristearate, beeswax, lecithin, or resins, such as, for example but not limited to, dammar gum, mastic gum, copal gum, shellac, to enhance mechanical properties.

In various embodiments, the wax can be diluted in peanut oil, sunflower oil, soybean oil canola oil, olive oil, or rice bran oil or any combination thereof.

In various embodiments, the wax can be prepared by transferring it into a container, heating them, then using them. In various embodiments, the wax can be melted at a higher temperature to use in an injection process at high temperature in an oven. In some embodiments, the wax can be processed at room temperature. In various embodiments, the wax can be combined with another wax to obtain desired physical, mechanical, and/or chemical properties. In some instances, more than one wax is combined to warm or to liquify before mixing them.

In various embodiments, the wax can be prepared using a casting process at a temperature range between about 4° C. and about 40° C. In various embodiments, the initial structure hydrogel can be incubated for solvent exchange for improved filling of the negative space by wax. In various embodiments, the hydrogel can be incubated in acetone or tetrahydrofuran or any combinations thereof. In various embodiments, the negative space of the initial structure is exposed to a lipophilic agent. In various embodiments, the lipophilic agent can be acetone or tetrahydrofuran.

In various embodiments, the cast structure contains a gallium or gallium containing material, such as, for example but not limited to, gallium-indium, gallium-tin, and gallium-indium-tin. In various embodiments, the cast structure has a surface roughness (Ra) less than 10 μm, 5 μm, 3 μm, 2 μm, 1 μm, 0.8 μm, 0.6 μm, 0.5 μm, 0.4 μm, 0.3 μm, 0.2 μm, or 0.1 μm. In various embodiments, the cast structure is post-processed by metallurgical techniques to provide additional features, including features with other surface properties. In various embodiments, where the cast structure includes independent vascular networks, the vascular networks can be post-processed independently. In various embodiments, where one network can be connected to an electrode and submerged in an electroplating bath and additional metal can be electroplated on top. In various embodiments, the electroplating is directionally applied and more at distal than proximal. In various embodiments, the electroplating is uniformly applied evenly over the surface. In various embodiments, the electroplating is the same metal or can be a different metal from the cast structure.

In various embodiments, dip coating can be used to modify the cast structure. In various embodiments, dip coating can be performed in sequential materials. In various embodiments, dip coating can be performed in solutions, which is then dried and solidified. In various embodiments, dip coating can be performed in solutions, which is then crystallized. In various embodiments, dip coating can be performed by nanometer-thick polyelectrolyte polymer films layer by layer. In various embodiments, dip coating can be performed in suspension of magnetic particles, magnetic field can align or control the surface changes. In various embodiments, the independent vascular networks are electrically charged and followed by powder casting. In various embodiments, the process includes laminin first, then follows by dip coating in collagen. In some embodiments, the process does not include a hard bake, such as required in those processes used in powder coating of parts for automobiles. In various embodiments, the cast structure can be coated using organic polymer in alternating layers (OPAL) process, where each coating layer can include a different material, which may give rise to a different color, along one or more directions on the surface and/or along the growth direction of the cast structure, which may form a series of colors (e.g., rainbow) across the surface. In other words, the cast structure can be coated to have a gradient in the coating by controlled mixing in the structure in both 2D and/or 3D arrangements of the coating, along a surface and/or through the layer of the coating.

In various embodiments, the gallium or gallium containing material, such as, for example but not limited to, gallium-indium, gallium-tin, and gallium-indium-tin, can be removed via electrophoresis. The remaining amount of the gallium content after removal can be determined via elemental analysis. In various embodiments, there may be less gallium content in the coating of the cast structure than before the coating is performed.

In various embodiments, the initial structure can include a hydrogel, where the hydrogel is alginate. In various embodiments, the alginate can be crosslinked with calcium chloride. In various embodiments, the alginate can be dissolved with a calcium chelator. In various embodiments, the chelator can be sodium citrate or ethylenediaminetetraacetic acid (EDTA).

In various embodiments, the initial structure can include hydrogel components. In various embodiments, the state of matter of the injected material can be altered by pH or salt concentration. In various embodiments, the injected material can involve formation of material through multivalent inorganic cations or anions. In various embodiments, the injected material can be a polysaccharide such as alginate, wherein the alginate can be crosslinked with calcium divalent cations from calcium chloride.

In various embodiments, the cast structure can be removed through the use of a cationic or anionic chelator. In various embodiments, the chemical used to dissolve the cast structure is a calcium chelator. In various embodiments, the chelator can be sodium citrate or EDTA. In various embodiments, the injected material can involve formation of material through disulfide bonds. In various embodiments, the cast structure can be removed under reducing conditions. In various embodiments, the injected material can involve a covalently adaptable network. In various embodiments, the adaptable bond can involve formation of a reversible thioester. In various embodiments, the injected material can involve a thermodynamically reversible network. In various embodiments, the thermodynamically reversible network can be formed by coupling of a furan and maleimide. In various embodiments, the thermodynamically reversible network can be initiated and reversed using heat/temperature. In various embodiments, the Diels Alder adduct product can be formed at 60° C. In various embodiments, the retro Diels Alder product can be formed at 110° C.

In various embodiments, the cast structure can be agarose. In various embodiments, the cast structure can be a biologically-derived polymeric material, wherein the material is gelatin. In various embodiments, the gelatin can be crosslinked by an enzymatic or chemical crosslinker. In various embodiments, the enzymatic crosslinker can be microbial transglutaminase. In various embodiments, the chemical crosslinker can be glutaraldehyde. In various embodiments, the gelatin cast can be removed by enzymatic degradation. In various embodiments, the enzyme can be collagenase.

In accordance with various embodiments, the method S100 includes, at step S108, removing the initial structure from the cast material. In various embodiments, the initial structure can be removed via a number of processes and techniques, including for example, but not limited to, by dissolution or degradation, by one or more chemical processes under acidic or basic conditions, collagenase incubation with a protease or peptidase, including trypsin, collagenase, proteinase k, cathepsin K, or any combination thereof, or by one or more physical processes via a mechanical process, swelling, drying, heating, or via a light process due to presence of photolabile linkers in the initial structure, or any combination thereof. In various embodiments, metals that are liquid (e.g., liquid metal) at room or ambient temperature, but solid at lower temperatures, dissolution or degradation can be performed at low temperature, such as below 0° C. (273 Kelvin), by incubating the initial structure in a dissolution or degradation solution containing glycerol. In various embodiments, metals that are liquid at temperatures, e.g., up to 37° C., but solid at room temperatures or lower temperatures, dissolution or degradation can be performed below the room or ambient temperatures.

In accordance with various embodiments, the method S100 includes, at step S110, obtaining a final structure comprising the cast material. In various embodiments, the final structure can be an artificial tissue or organ, a tissue model, a phantom, or a stent. In various embodiments, the final structure can be a vascular cast.

In accordance with various embodiments, the method S100 optionally includes, at step S112, modifying a surface of the final structure. In various embodiments, modifying the surface of the final structure can comprise roughening or smoothing of the surface. In various embodiments, modifying the surface of the final structure can comprise coating with a layer of material comprising, but not limited to, a biocompatible material, a hemocompatible material, cells, a cell-adhesive material, collagen, gelatin, fibronectin, laminin, polylysine, PEG based peptide conjugates, polyurethane, or any combination thereof, or a metal comprising copper, nickel, or gold. In various embodiments, modifying the surface of the final structure can comprise selectively coating a portion of the surface with a material comprising, but not limited to, a biocompatible material, a hemocompatible material, cells, a cell-adhesive material, collagen, gelatin, fibronectin, laminin, polylysine, PEG based peptide conjugates, polyurethane, or any combination thereof, or a metal comprising copper, nickel, or gold. In various embodiments, modifying the surface of the final structure can comprise any of electroplating, performing electrolysis, using ferro fluid in magnetic field to perform a surface treatment of the surface, or any combination thereof.

In various embodiments, the cast structure can be prepared for coating by lowering surface tension so that droplets do not form on the surface of the cast structure. In various embodiments, a coating material of the cast structure can contain a lipid. In various embodiments, the lipid can be a phospholipid, steroid, glycolipid, sphingolipid, or amphiphile or any combination thereof. In various embodiments, the phospholipid can be a zwitterionic phospholipid, or anionic phospholipid, or PEGylated phospholipid or any combination thereof. In various embodiments, the lipid can be polylactic acid. In various embodiments, the lipid can be a triglyceride. In various embodiments, the coating material can contain hydrophobic regions, wherein the hydrophobic containing material is a peptide or protein. In various embodiments, the material can be serum. In various embodiments, the hydrophobic material can be a lipid. In various embodiments, the coated structure allows for cell adhesion. In various embodiments, the cast structure is coated with a material to enable full removal of the first material in downstream applications, and the material is pluronic.

In accordance with various embodiments, the method S100 optionally includes, at step S114, forming one or more coatings on the final structure. In various embodiments, the one or more coatings can include a biocompatible material, a hemocompatible material, cells, a cell-adhesive material, collagen, gelatin, fibronectin, laminin, polylysine, PEG based peptide conjugates, polyurethane, or any combination thereof, or a metal comprising copper, nickel, or gold.

In various embodiments, the one or more coatings has a thickness between 10 nm and 1000 μm. In various embodiments, the one or more coatings can include a multilayer coating, wherein the multilayer coating is obtained by forming a coating at least two times. In various embodiments, the multilayer coating on the final structure comprises at least two different coating materials.

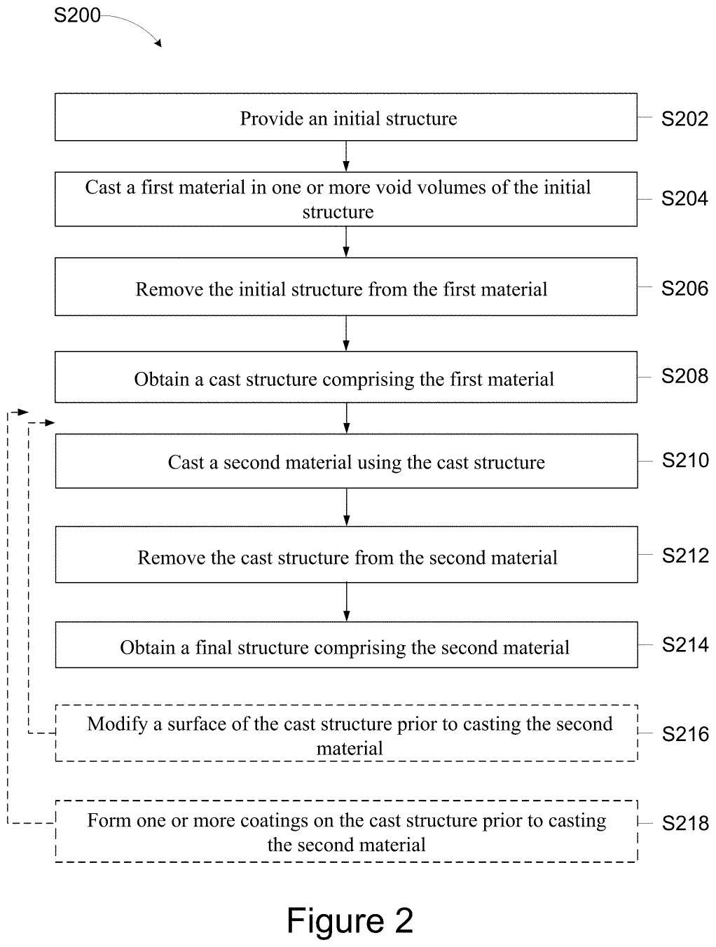

FIG. 2 is a flowchart for a method S200 for preparing a structure, according to various embodiments. In accordance with various embodiments, the method S200 includes, at step S202, providing an initial structure. In various embodiments, the initial structure can be printed and can have one or more features. In various embodiments, the one or more features can be positive or negative features. In accordance with various embodiments, the positive features refer to spaces that are occupied by a material that made up the structure. In accordance with various embodiments, the negative features refer to spaces that are absent of a material that made up the structure, e.g., a void space or a void volume within the structure. In accordance with various embodiments, the one or more negative features refer to one or more void volumes of the structure.

In various embodiments, the initial structure is a 3-D hydrogel structure that is made of a hydrogel matrix. In various embodiments, the initial structure can include a wax, a plastic, or polyvinyl alcohol, PEGDA having 250-35,000 Da, PEG-norbornene, PEG-MMP, gelatin methacrylate, or any combination thereof. In various embodiments, one or more features comprises a vascular topology that is generated by additive or subtractive manufacturing. In various embodiments, the initial structure can be printed via a 3-D printer. In various embodiments, the initial structure can include a vascular topology within a tissue or organ.

In various embodiments, after the initial structure is printed, a post-process can be performed on the initial structure. In various embodiments, the post-process can include, among many others, washing the initial structure in a solvent, equilibrating the initial structure in a solvent, or crosslinking the initial structure in a lightbox.

In accordance with various embodiments, the method S200 includes, at step S204, casting a first material in one or more void volumes of the initial structure. In various embodiments, the first material (e.g., cast material) can include, but not limited to, a thermoreversible material, metal or liquid metal including gallium, alloys including Field's metal, gallium-indium, gallium-tin, and gallium-indium-tin, supercooled liquid metal, carbohydrate glass, pluronic, low molecular weight PEG, PCL (polycaprolactone), gelatin, wax, or any combination thereof.

In various embodiments, the wax for the initial structure or the first/cast material can be a wax from a diverse class of organic compounds. In some embodiments, the wax can be lipophilic and/or malleable solids near ambient temperatures. In various embodiments, the wax can include higher alkanes and lipids, with melting points above about 40° C. (104° F.), melting to give low viscosity liquids. In various embodiments, the wax can be insoluble in water but soluble in organic, nonpolar solvents.

In various embodiments, the wax can be an alkane hydrocarbon, where the alkane hydrocarbon has a formula CH₃(CH₂)ₙCH₃, where n=14-48. In various embodiments, the alkane hydrocarbon can be hexadecane, heptadecane, octadecane, eicosane, heneicosane, docosane, tetracosane, nonacosane, triacontane, hentriacontane, dotriacontane, hexatriacontane, tetracontane, tetratetracontane, or pentacontane. In various embodiments, the wax can be an alkene hydrocarbon, where the alkene hydrocarbon has a formula CH₃(CH₂)ₙCH═CH₂, where n=14-48. In various embodiments, the alkene hydrocarbon can be 1-octadecene.

In various embodiments, the wax can be an unsaturated hydrocarbon fatty alcohol.

In various embodiments, the wax can be a saturated hydrocarbon fatty alcohol, where the saturated hydrocarbon can be decyl alcohol, lauryl alcohol, myristyl alcohol, palmityl alcohol, stearyl alcohol, arachidyl alcohol, behenyl alcohol, lignoceryl alcohol, ceryl alcohol, myricyl alcohol, or any combination thereof.

In various embodiments, the wax can be a lipid that contains saturated fatty acid, where the fatty acid has a formula CH₃(CH₂)ₙCOOH, where n=4-30. In various embodiments, the lipid can be hexanoic acid, nonanoic acid, octanoic acid, decanoic acid, methyl nonadecanoate, lauric acid, methyl arachidate, myristic acid, methyl tricosanoate, methyl behenate, heptadecanoic acid, palmitic acid, stearic acid, or melissic acid or any combination thereof.

In various embodiments, the wax can be a lipid that contains an unsaturated fatty acid, where the lipid is arachidonic acid, linolenic acid, palmitoleic acid, oleic acid, or any combinations thereof. In various embodiments, the wax can be a lipid combination that contains multiple saturated fatty acids or unsaturated fatty acids. In various embodiments, the wax can be a lipid combination that contains a saturated fatty acid or an unsaturated fatty acid.

In various embodiments, the wax is plant derived, animal derived, petroleum derived, or synthetic or any combination thereof.

In various embodiments, the wax can be paraffin, soy wax, beeswax (major component is myricyl palmitate), gulf wax, carnauba wax, candelilla wax, polyethylene wax, microcrystalline wax, or any combination thereof.

In various embodiments, the wax can contain additives, such as, for example but not limited to, stearic acid, glyceryl tristearate, sorbitan tristearate, beeswax, lecithin, or resins, such as, for example but not limited to, dammar gum, mastic gum, copal gum, shellac, to enhance mechanical properties.

In various embodiments, the wax can be diluted in peanut oil, sunflower oil, soybean oil canola oil, olive oil, or rice bran oil or any combination thereof.

In various embodiments, the wax can be prepared by transferring it into a container, heating them, then using them. In various embodiments, the wax can be melted at a higher temperature to use in an injection process at high temperature in an oven. In some embodiments, the wax can be processed at room temperature. In various embodiments, the wax can be combined with another wax to obtain desired physical, mechanical, and/or chemical properties. In some instances, more than one wax is combined to warm or to liquify before mixing them.

In various embodiments, the wax can be prepared using a casting process at a temperature range between about 4° C. and about 40° C. In various embodiments, the initial structure hydrogel can be incubated for solvent exchange for improved filling of the negative space by wax. In various embodiments, the hydrogel can be incubated in acetone or tetrahydrofuran or any combinations thereof. In various embodiments, the negative space of the initial structure is exposed to a lipophilic agent. In various embodiments, the lipophilic agent can be acetone or tetrahydrofuran.

In various embodiments, the cast structure contains a gallium or gallium containing material, such as, for example but not limited to, gallium-indium, gallium-tin, and gallium-indium-tin. In various embodiments, the cast structure has a surface roughness (Ra) less than 10 μm, 5 μm, 3 μm, 2 μm, 1 μm, 0.8 μm, 0.6 μm, 0.5 μm, 0.4 μm, 0.3 μm, 0.2 μm, or 0.1 μm. In various embodiments, the cast structure is post-processed by metallurgical techniques to provide additional features, including features with other surface properties. In various embodiments, where the cast structure includes independent vascular networks, the vascular networks can be post-processed independently. In various embodiments, where one network can be connected to an electrode and submerged in an electroplating bath and additional metal can be electroplated on top. In various embodiments, the electroplating is directionally applied and more at distal than proximal. In various embodiments, the electroplating is uniformly applied evenly over the surface. In various embodiments, the electroplating is the same metal or can be a different metal from the cast structure.

In various embodiments, dip coating can be used to modify the cast structure. In various embodiments, dip coating can be performed in sequential materials. In various embodiments, dip coating can be performed in solutions, which is then dried and solidified. In various embodiments, dip coating can be performed in solutions, which is then crystallized. In various embodiments, dip coating can be performed by nanometer-thick polyelectrolyte polymer films layer by layer. In various embodiments, dip coating can be performed in suspension of magnetic particles, magnetic field can align or control the surface changes. In various embodiments, the independent vascular networks are electrically charged and followed by powder casting. In various embodiments, the process includes laminin first, then follows by dip coating in collagen. In some embodiments, the process does not include a hard bake, such as required in those processes used in powder coating of parts for automobiles. In various embodiments, the cast structure can be coated using organic polymer in alternating layers (OPAL) process, where each coating layer can include a different material, which may give rise to a different color, along one or more directions on the surface and/or along the growth direction of the cast structure, which may form a series of colors (e.g., rainbow) across the surface. In other words, the cast structure can be coated to have a gradient in the coating by controlled mixing in the structure in both 2D and/or 3D arrangements of the coating, along a surface and/or through the layer of the coating.

In various embodiments, the gallium or gallium containing material, such as, for example but not limited to, gallium-indium, gallium-tin, and gallium-indium-tin, can be removed via electrophoresis. The remaining amount of the gallium content after removal can be determined via elemental analysis. In various embodiments, there may be less gallium content in the coating of the cast structure than before the coating is performed.

In various embodiments, the initial structure can include a hydrogel, where the hydrogel is alginate. In various embodiments, the alginate can be crosslinked with calcium chloride. In various embodiments, the alginate can be dissolved with a calcium chelator. In various embodiments, the chelator can be sodium citrate or ethylenediaminetetraacetic acid (EDTA).

In various embodiments, the initial structure can include hydrogel components. In various embodiments, the state of matter of the injected material can be altered by pH or salt concentration. In various embodiments, the injected material can involve formation of material through multivalent inorganic cations or anions. In various embodiments, the injected material can be a polysaccharide such as alginate, wherein the alginate can be crosslinked with calcium divalent cations from calcium chloride.

In various embodiments, the cast structure can be removed through the use of a cationic or anionic chelator. In various embodiments, the chemical used to dissolve the first material is a calcium chelator. In various embodiments, the chelator can be sodium citrate or EDTA. In various embodiments, the injected material can involve formation of material through disulfide bonds. In various embodiments, the cast structure can be removed under reducing conditions. In various embodiments, the injected material can involve a covalently adaptable network. In various embodiments, the adaptable bond can involve formation of a reversible thioester. In various embodiments, the injected material can involve a thermodynamically reversible network. In various embodiments, the thermodynamically reversible network can be formed by coupling of a furan and maleimide. In various embodiments, the thermodynamically reversible network can be initiated and reversed using heat/temperature. In various embodiments, the Diels Alder adduct product can be formed at 60° C. In various embodiments, the retro Diels Alder product can be formed at 110° C.

In various embodiments, the first material can be agarose. In various embodiments, the first material can be a biologically-derived polymeric material, wherein the material is gelatin. In various embodiments, the gelatin can be crosslinked by an enzymatic or chemical crosslinker. In various embodiments, the enzymatic crosslinker can be microbial transglutaminase. In various embodiments, the chemical crosslinker can be glutaraldehyde. In various embodiments, the gelatin cast can be removed by enzymatic degradation. In various embodiments, the enzyme can be collagenase.

In various embodiments, casting the first material in the one or more void volumes of the initial structure includes: filling the first material in the one or more void volumes of the initial structure at a first temperature; and solidifying the first material at a second temperature, wherein the first material at the first temperature and the first material at the second temperature have different physical states. In accordance with various embodiments, the first temperature for the first material, such as gallium and other liquid metals, ranges from about 65° C. to about 4° C., and for the first material, such as PCL, ranges from about 65° C. to about 35° C. In various embodiments, the second temperature for the first material, such as gallium and other liquid metals, and PCL, ranges from room temperature to about 4° C. In some embodiments, the cast material filling/solidification is not temperature sensitive and can be performed between about 37° C. and about 4° C.

In various embodiments, casting the first material in the one or more void volumes of the initial structure comprises: injecting the first material in the one or more void volumes of the initial structure at a first temperature; supercooling the first material; and crystalizing the first material at a second temperature. In various embodiments, the first material (e.g., cast material) can be a super cooled material. For example, gallium, which is solid at room temperature but liquid above 30° C. temperature, can be incubated at 37° C. temperature to liquify it, then incubated at 4° C. or lower to supercool it, then the supercooled gallium can be cast into the initial structure at room temperature (e.g., 21° C.). In various embodiments, a solid piece of gallium is physically connected to the supercooled gallium, resulting in crystallization of the supercooled gallium to solidify it at room temperature. In various embodiments, the supercooled gallium injected at room temperature into the initial structure can be warmed to 37° C., then placed at room temperature to solidify it.

In accordance with various embodiments, the method S200 includes, at step S206, removing the initial structure from the first material. In various embodiments, the initial structure can be removed via a number of processes and techniques, including for example, but not limited to, by dissolution or degradation, by one or more chemical processes under acidic or basic conditions, collagenase incubation with a protease or peptidase, including trypsin, collagenase, proteinase k, cathepsin K, or any combination thereof, or by one or more physical processes via a mechanical process, swelling, drying, heating, or via a light process due to presence of photolabile linkers in the initial structure, or any combination thereof. In various embodiments, metals that are liquid (e.g., liquid metal) at room or ambient temperature, but solid at lower temperatures, dissolution or degradation can be performed at low temperature, such as below 0° C. (273 Kelvin), by incubating the initial structure in a dissolution or degradation solution containing glycerol.

In accordance with various embodiments, the method S200 includes, at step S208, obtaining a cast structure comprising the first material (e.g., cast material). In various embodiments, the cast structure comprises a negative or inverted mold of the initial structure.

In accordance with various embodiments, the method S200 includes, at step S210, casting a second material using the cast structure. In various embodiments, the second material can include, but not limited to, a biomaterial comprising silk, collagen, gelatin, fibrin, synthetic peptides, hyaluronic acid, polymers comprising alginate, polyurethane, polycaprolactone (PCL), elastomers, collagen methacrylate, collagen methacrylamide, gelatin methacrylate, gelatin methacrylamide, silk methacrylate, silk methacrylamide, hyaluronic acid methacrylate, hyaluronic acid methacrylamide, pluronic diacrylate, pluronic methacrylamide, chondroitin sulfate methacrylate, chondroitin sulfate methacrylamide, elastin methacrylate, elastin methacrylamide, cellulose acrylate, cellulose methacrylamide, dextran methacrylate, dextran methacrylamide, heparin methacrylate, heparin methacrylamide, N-isopropyl acrylamide (NIPAAm), chitosan methacrylate, chitosan methacrylamide, polyethylene glycol norbornene, polyethylene glycol dithiol, thiolated gelatin, thiolated chitosan, thiolated hyaluronic acid, thiolated silk, PEG based peptide conjugates, decellularized ECM of any tissue/organ, plastic, metal, including gallium or indium, or alloys, including Field's metal, gallium-indium, gallium-tin, and gallium-indium-tin, supercooled liquid metal, or any combination thereof.

In accordance with various embodiments, the method S200 includes, at step S212, removing the cast structure from the second material. In various embodiments, the cast structure is removed by dissolution or degradation, by one or more chemical processes under acidic or basic conditions (e.g., under reducing conditions (in the case of disulfide bonds)), collagenase incubation with a protease or peptidase, including trypsin, collagenase, proteinase k, cathepsin K, or any combination thereof, or by one or more physical processes via a mechanical process, swelling, heating the cast structure to liquify the first material, or a light process due to presence of photolabile linkers in the initial structure, or any combination thereof. In various embodiments, metals that are liquid (e.g., liquid metal) at room or ambient temperature, but solid at lower temperatures, dissolution or degradation can be performed at low temperature, such as below 0° C. (273 Kelvin), by incubating the initial structure in a dissolution or degradation solution containing glycerol. In various embodiments, the cast structure can be removed by cooling. In various embodiments, the cast structure can be removed by adjusting the pH, for example, if alginate is used as the cast material; if PEGDA is used as the first material (for example, the initial structure is composed of GelMA, PEGDA is the first material cast into the void, the initial structure is enzymatically degraded, then a plastic is used as the second material for casting, and finally the first material PEGDA is degraded with sodium hydroxide). In various embodiments, the cast structure can be removed by enzymes (trypsin, collagenase, etc.) if gelatin/GelMA material is used as the first material (for example, the initial structure is composed of PEGDA, gelatin/GelMA is the first material cast into the void, the initial structure is removed by hydrolytic dissolution or degradation, PEG-norbornene is used as the second material to cast, and finally the first material is removed by enzymatic dissolution or degradation).

In accordance with various embodiments, the method S200 includes, at step S214, obtaining a final structure comprising the second material. In various embodiments, the final structure can be an artificial tissue or organ.

In accordance with various embodiments, the method S200 optionally includes, at step S216, modifying a surface of the cast structure prior to casting the second material at step S210. In various embodiments, modifying the surface of the cast structure further comprises roughening or smoothing of the surface, coating with a layer of collagen, coating a portion of the surface with collagen, electroplating, performing electrolysis, using ferro fluid in magnetic field to perform a surface treatment of the surface, or any combination thereof.

In various embodiments, the cast structure can be prepared for coating by lowering surface tension so that droplets do not form on the surface of the cast structure. In various embodiments, a coating material of the cast structure can contain a lipid. In various embodiments, the lipid can be a phospholipid, steroid, glycolipid, sphingolipid, or amphiphile or any combination thereof. In various embodiments, the phospholipid can be a zwitterionic phospholipid, or anionic phospholipid, or PEGylated phospholipid or any combination thereof. In various embodiments, the lipid can be polylactic acid. In various embodiments, the lipid can be a triglyceride. In various embodiments, the coating material can contain hydrophobic regions, wherein the hydrophobic containing material is a peptide or protein. In various embodiments, the material can be serum. In various embodiments, the hydrophobic material can be a lipid. In various embodiments, the coated structure allows for cell adhesion. In various embodiments, the cast structure is coated with a material to enable full removal of the first material in downstream applications, and the material is pluronic.

In accordance with various embodiments, the method S200 optionally includes, at step S218, forming one or more coatings on the cast structure prior to casting the second material at step S210. In various embodiments, the one or more coatings can include a biocompatible material, a hemocompatible material, cells, a cell-adhesive material, collagen, gelatin, fibronectin, laminin, polylysine, PEG based peptide conjugates, polyurethane, or any combination thereof, or a metal comprising copper, nickel, or gold.

In various embodiments, the one or more coatings has a thickness between 10 nm and 1000 μm. In various embodiments, the one or more coatings can include a multilayer coating, wherein the multilayer coating is obtained by forming a coating at least two times. In various embodiments, the multilayer coating on the final structure comprises at least two different coating materials.

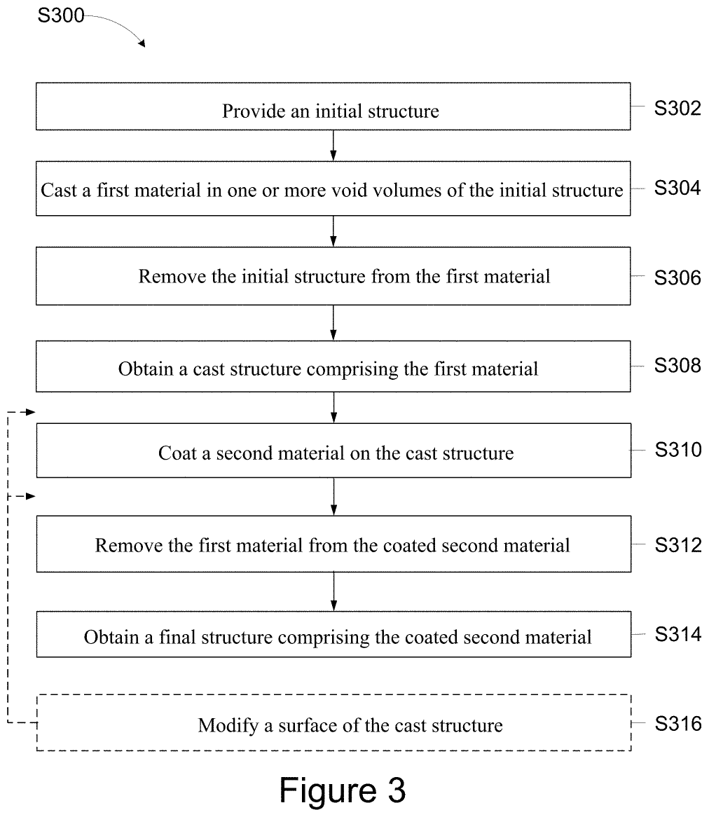

FIG. 3 is a flowchart for a method S300 for preparing a structure, according to various embodiments. In accordance with various embodiments, the method S300 includes, at step S302, providing an initial structure. In various embodiments, the initial structure can be printed and can have one or more features. In various embodiments, the one or more features can be positive or negative features. In accordance with various embodiments, the positive features refer to spaces that are occupied by a material that made up the structure. In accordance with various embodiments, the negative features refer to spaces that are absent of a material that made up the structure, e.g., a void space or a void volume within the structure. In accordance with various embodiments, the one or more negative features refer to one or more void volumes of the structure.

In various embodiments, the initial structure is a 3-D hydrogel structure that is made of a hydrogel matrix. In various embodiments, the initial structure can include a wax, a plastic, or polyvinyl alcohol, PEGDA having 250-35,000 Da, PEG-norbornene, PEG-MMP, gelatin methacrylate, or any combination thereof. In various embodiments, one or more features comprises a vascular topology that is generated by additive or subtractive manufacturing. In various embodiments, the initial structure can be printed via a 3-D printer. In various embodiments, the initial structure can include a vascular topology within a tissue or organ.

In various embodiments, after the initial structure is printed, a post-process can be performed on the initial structure. In various embodiments, the post-process can include, among many others, washing the initial structure in a solvent, equilibrating the initial structure in a solvent, or crosslinking the initial structure in a lightbox.

In accordance with various embodiments, the method S300 includes, at step S304, casting a first material in one or more void volumes of the initial structure. In various embodiments, the first material (e.g., cast material) can include, but not limited to, a thermoreversible material, metal or liquid metal including gallium, alloys including Field's metal, gallium-indium, gallium-tin, and gallium-indium-tin, carbohydrate glass, pluronic, low molecular weight PEG, PCL, gelatin, wax, or any combination thereof.

In various embodiments, the wax for the initial structure or the first/cast material can be a wax from a diverse class of organic compounds. In some embodiments, the wax can be lipophilic and/or malleable solids near ambient temperatures. In various embodiments, the wax can include higher alkanes and lipids, with melting points above about 40° C. (104° F.), melting to give low viscosity liquids. In various embodiments, the wax can be insoluble in water but soluble in organic, nonpolar solvents.

In various embodiments, the wax can be an alkane hydrocarbon, where the alkane hydrocarbon has a formula CH₃(CH₂)ₙCH₃, where n=14-48. In various embodiments, the alkane hydrocarbon can be hexadecane, heptadecane, octadecane, eicosane, heneicosane, docosane, tetracosane, nonacosane, triacontane, hentriacontane, dotriacontane, hexatriacontane, tetracontane, tetratetracontane, or pentacontane. In various embodiments, the wax can be an alkene hydrocarbon, where the alkene hydrocarbon has a formula CH₃(CH₂)ₙCH═CH₂, where n=14-48. In various embodiments, the alkene hydrocarbon can be 1-octadecene.

In various embodiments, the wax can be an unsaturated hydrocarbon fatty alcohol. In various embodiments, the wax can be a saturated hydrocarbon fatty alcohol, where the saturated hydrocarbon can be decyl alcohol, lauryl alcohol, myristyl alcohol, palmityl alcohol, stearyl alcohol, arachidyl alcohol, behenyl alcohol, lignoceryl alcohol, ceryl alcohol, myricyl alcohol, or any combination thereof.

In various embodiments, the wax can be a lipid that contains saturated fatty acid, where the fatty acid has a formula CH₃(CH₂)ₙCOOH, where n=4-30. In various embodiments, the lipid can be hexanoic acid, nonanoic acid, octanoic acid, decanoic acid, methyl nonadecanoate, lauric acid, methyl arachidate, myristic acid, methyl tricosanoate, methyl behenate, heptadecanoic acid, palmitic acid, stearic acid, or melissic acid or any combination thereof.

In various embodiments, the wax can be a lipid that contains an unsaturated fatty acid, where the lipid is arachidonic acid, linolenic acid, palmitoleic acid, oleic acid, or any combinations thereof. In various embodiments, the wax can be a lipid combination that contains multiple saturated fatty acids or unsaturated fatty acids. In various embodiments, the wax can be a lipid combination that contains a saturated fatty acid or an unsaturated fatty acid.

In various embodiments, the wax is plant derived, animal derived, petroleum derived, or synthetic or any combination thereof.

In various embodiments, the wax can be paraffin, soy wax, beeswax (major component is myricyl palmitate), gulf wax, carnauba wax, candelilla wax, polyethylene wax, microcrystalline wax, or any combination thereof.

In various embodiments, the wax can contain additives, such as, for example but not limited to, stearic acid, glyceryl tristearate, sorbitan tristearate, beeswax, lecithin, or resins, such as, for example but not limited to, dammar gum, mastic gum, copal gum, shellac, to enhance mechanical properties.

In various embodiments, the wax can be diluted in peanut oil, sunflower oil, soybean oil canola oil, olive oil, or rice bran oil or any combination thereof.

In various embodiments, the wax can be prepared by transferring it into a container, heating them, then using them. In various embodiments, the wax can be melted at a higher temperature to use in an injection process at high temperature in an oven. In some embodiments, the wax can be processed at room temperature. In various embodiments, the wax can be combined with another wax to obtain desired physical, mechanical, and/or chemical properties. In some instances, more than one wax is combined to warm or to liquify before mixing them.

In various embodiments, the wax can be prepared using a casting process at a temperature range between about 4° C. and about 40° C. In various embodiments, the initial structure hydrogel can be incubated for solvent exchange for improved filling of the negative space by wax. In various embodiments, the hydrogel can be incubated in acetone or tetrahydrofuran or any combinations thereof. In various embodiments, the negative space of the initial structure is exposed to a lipophilic agent. In various embodiments, the lipophilic agent can be acetone or tetrahydrofuran.

In various embodiments, the cast structure contains a gallium or gallium containing material, such as, for example but not limited to, gallium-indium, gallium-tin, and gallium-indium-tin. In various embodiments, the cast structure has a surface roughness (Ra) less than 10 μm, 5 μm, 3 μm, 2 μm, 1 μm, 0.8 μm, 0.6 μm, 0.5 μm, 0.4 μm, 0.3 μm, 0.2 μm, or 0.1 μm. In various embodiments, the cast structure is post-processed by metallurgical techniques to provide additional features, including features with other surface properties. In various embodiments, where the cast structure includes independent vascular networks, the vascular networks can be post-processed independently. In various embodiments, where one network can be connected to an electrode and submerged in an electroplating bath and additional metal can be electroplated on top. In various embodiments, the electroplating is directionally applied and more at distal than proximal. In various embodiments, the electroplating is uniformly applied evenly over the surface. In various embodiments, the electroplating is the same metal or can be a different metal from the cast structure.

In various embodiments, dip coating can be used to modify the cast structure. In various embodiments, dip coating can be performed in sequential materials. In various embodiments, dip coating can be performed in solutions, which is then dried and solidified. In various embodiments, dip coating can be performed in solutions, which is then crystallized. In various embodiments, dip coating can be performed by nanometer-thick polyelectrolyte polymer films layer by layer. In various embodiments, dip coating can be performed in suspension of magnetic particles, magnetic field can align or control the surface changes. In various embodiments, the independent vascular networks are electrically charged and followed by powder casting. In various embodiments, the process includes laminin first, then follows by dip coating in collagen. In some embodiments, the process does not include a hard bake, such as required in those processes used in powder coating of parts for automobiles. In various embodiments, the cast structure can be coated using organic polymer in alternating layers (OPAL) process, where each coating layer can include a different material, which may give rise to a different color, along one or more directions on the surface and/or along the growth direction of the cast structure, which may form a series of colors (e.g., rainbow) across the surface. In other words, the cast structure can be coated to have a gradient in the coating by controlled mixing in the structure in both 2D and/or 3D arrangements of the coating, along a surface and/or through the layer of the coating.

In various embodiments, the gallium or gallium containing material, such as, for example but not limited to, gallium-indium, gallium-tin, and gallium-indium-tin, can be removed via electrophoresis. The remaining amount of the gallium content after removal can be determined via elemental analysis. In various embodiments, there may be less gallium content in the coating of the cast structure than before the coating is performed.

In various embodiments, the initial structure can include a hydrogel, where the hydrogel is alginate. In various embodiments, the alginate can be crosslinked with calcium chloride. In various embodiments, the alginate can be dissolved with a calcium chelator. In various embodiments, the chelator can be sodium citrate or ethylenediaminetetraacetic acid (EDTA).

In various embodiments, the initial structure can include hydrogel components. In various embodiments, the state of matter of the injected material can be altered by pH or salt concentration. In various embodiments, the injected material can involve formation of material through multivalent inorganic cations or anions. In various embodiments, the injected material can be a polysaccharide such as alginate, wherein the alginate can be crosslinked with calcium divalent cations from calcium chloride.

In various embodiments, the cast structure can be removed through the use of a cationic or anionic chelator. In various embodiments, the chemical used to dissolve the first material is a calcium chelator. In various embodiments, the chelator can be sodium citrate or EDTA. In various embodiments, the injected material can involve formation of material through disulfide bonds. In various embodiments, the cast structure can be removed under reducing conditions. In various embodiments, the injected material can involve a covalently adaptable network. In various embodiments, the adaptable bond can involve formation of a reversible thioester. In various embodiments, the injected material can involve a thermodynamically reversible network. In various embodiments, the thermodynamically reversible network can be formed by coupling of a furan and maleimide. In various embodiments, the thermodynamically reversible network can be initiated and reversed using heat/temperature. In various embodiments, the Diels Alder adduct product can be formed at 60° C. In various embodiments, the retro Diels Alder product can be formed at 110° C.

In various embodiments, the first material can be agarose. In various embodiments, the first material can be a biologically-derived polymeric material, wherein the material is gelatin. In various embodiments, the gelatin can be crosslinked by an enzymatic or chemical crosslinker. In various embodiments, the enzymatic crosslinker can be microbial transglutaminase. In various embodiments, the chemical crosslinker can be glutaraldehyde. In various embodiments, the gelatin cast can be removed by enzymatic degradation. In various embodiments, the enzyme can be collagenase.

In various embodiments, casting the first material in the one or more void volumes of the initial structure includes: filling the first material in the one or more void volumes of the initial structure at a first temperature; and solidifying the first material at a second temperature, wherein the first material at the first temperature and the first material at the second temperature have different physical states. In accordance with various embodiments, the first temperature for the first material, such as gallium and other liquid metals, ranges from about 65° C. to about 4° C., and for the first material, such as PCL, ranges from about 65° C. to about 35° C. In various embodiments, the second temperature for the first material, such as gallium and other liquid metals, and PCL, ranges from room temperature to about 4° C. In some embodiments, the cast material filling/solidification is not temperature sensitive and can be performed between about 37° C. and about 4° C.

In accordance with various embodiments, the method S300 includes, at step S306, removing the initial structure from the first material. In various embodiments, the initial structure can be removed via a number of processes and techniques, including for example, but not limited to, by dissolution or degradation, by one or more chemical processes under acidic or basic conditions, collagenase incubation with a protease or peptidase, including trypsin, collagenase, proteinase k, cathepsin K, or any combination thereof, or by one or more physical processes via a mechanical process, swelling, drying, heating, or via a light process due to presence of photolabile linkers in the initial structure, or any combination thereof. In various embodiments, metals that are liquid (e.g., liquid metal) at room or ambient temperature, but solid at lower temperatures, dissolution or degradation can be performed at low temperature, such as below 0° C. (273 Kelvin), by incubating the initial structure in a dissolution or degradation solution containing glycerol. In various embodiments, the cast structure can be removed by cooling. In various embodiments, the cast structure can be removed by adjusting the pH, for example, if alginate is used as the cast material; if PEGDA is used as the cast material (GelMA gel, PEGDA void cast, enzymatic dissolution or degradation, plastic cast, sodium hydroxide dissolution or degradation). In various embodiments, the cast structure can be removed by enzymes (trypsin, collagenase, etc.) if gelatin/GelMA material is used as the cast material (PEGDA gel, GelMA void cast, hydrolytic dissolution or degradation, PEG-norbornene cast, collagenase dissolution or degradation).

In accordance with various embodiments, the method S300 includes, at step S308, obtaining a cast structure comprising the first material. In various embodiments, the cast structure comprises an inverted mold of the initial structure.

In accordance with various embodiments, the method S300 includes, at step S310, coating a second material on the cast structure. In various embodiments, the second material includes a biocompatible material, a hemocompatible material, cells, a cell-adhesive material, collagen, gelatin, fibronectin, laminin, polylysine, PEG based peptide conjugates, polyurethane, or any combination thereof, or a metal comprising copper, nickel, or gold.

In various embodiments, the second material is coated via dip-coating, spray coating, powder coating, vapor deposition, electroplating, or via oxidation and/or reduction reactions, or a combination thereof. In various embodiments, the second material is coated to a thickness between 10 nm and 1000 μm. In various embodiments, the one or more coatings can include a multilayer coating, wherein the multilayer coating is obtained by forming a coating at least two times. In various embodiments, the multilayer coating on the final structure comprises at least two different coating materials.

In accordance with various embodiments, the method S300 includes, at step S312, removing the first material (e.g., cast material) from the coated second material. In various embodiments, the first material is removed by dissolution or degradation, by one or more chemical processes under acidic or basic conditions, collagenase incubation with a protease or peptidase, including trypsin, collagenase, proteinase k, cathepsin K, or any combination thereof, or by one or more physical processes via a mechanical process, swelling, heating the cast structure to liquify the first material, or a light process due to presence of photolabile linkers in the initial structure, or any combination thereof. In various embodiments, metals that are liquid (e.g., liquid metal) at room or ambient temperature, but solid at lower temperatures, dissolution or degradation can be performed at low temperature, such as below 0° C. (273 Kelvin), by incubating the initial structure in a dissolution or degradation solution containing glycerol.

In accordance with various embodiments, the method S300 includes, at step S314, obtaining a final structure comprising the coated second material. In various embodiments, the final structure can be an artificial tissue or organ.

In accordance with various embodiments, the method S300 optionally includes, at step S316, modifying a surface of the cast structure prior to step S310 or modifying a surface of the coated second material prior to step 312. In various embodiments, modifying the surface of the cast structure further comprises roughening or smoothing of the surface, coating with a layer of collagen, coating a portion of the surface with collagen, electroplating, performing electrolysis, using ferro fluid in magnetic field to perform a surface treatment of the surface, or any combination thereof.

In various embodiments, the cast structure can be prepared for coating by lowering surface tension so that droplets do not form on the surface of the cast structure. In various embodiments, a coating material of the cast structure can contain a lipid. In various embodiments, the lipid can be a phospholipid, steroid, glycolipid, sphingolipid, or amphiphile or any combination thereof. In various embodiments, the phospholipid can be a zwitterionic phospholipid, or anionic phospholipid, or PEGylated phospholipid or any combination thereof. In various embodiments, the lipid can be polylactic acid. In various embodiments, the lipid can be a triglyceride. In various embodiments, the coating material can contain hydrophobic regions, wherein the hydrophobic containing material is a peptide or protein. In various embodiments, the material can be serum. In various embodiments, the hydrophobic material can be a lipid. In various embodiments, the coated structure allows for cell adhesion. In various embodiments, the cast structure is coated with a material to enable full removal of the first material in downstream applications, and the material is pluronic.

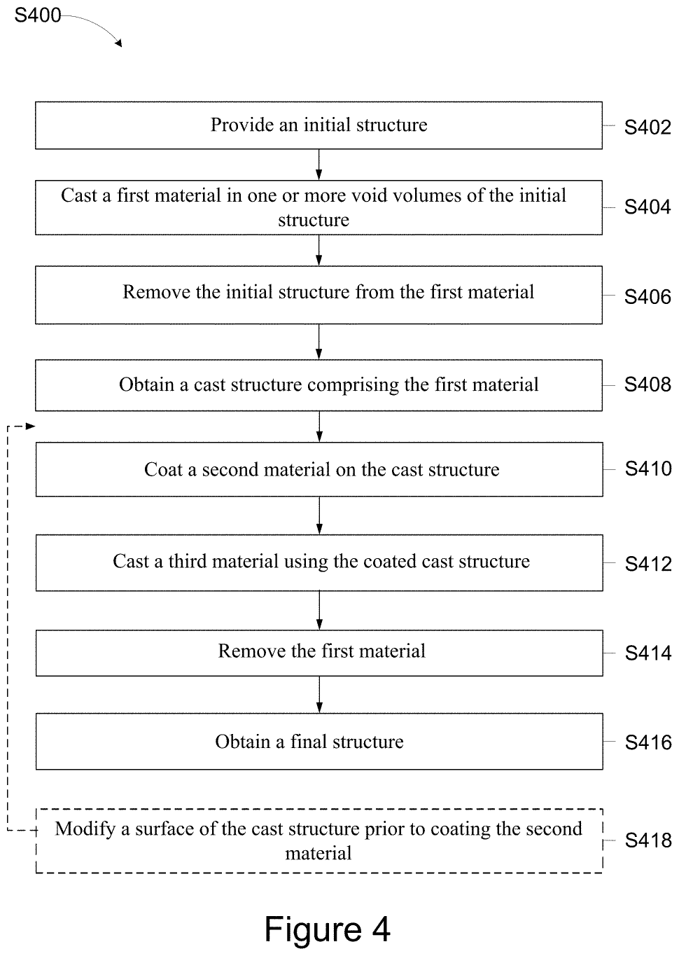

FIG. 4 is a flowchart for a method S400 for preparing a structure, according to various embodiments. In accordance with various embodiments, the method S400 includes, at step S402, providing an initial structure. In various embodiments, the initial structure can be printed and can have one or more features. In various embodiments, the one or more features can be positive or negative features. In accordance with various embodiments, the positive features refer to spaces that are occupied by a material that made up the structure. In accordance with various embodiments, the negative features refer to spaces that are absent of a material that made up the structure, e.g., a void space or a void volume within the structure. In accordance with various embodiments, the one or more negative features refer to one or more void volumes of the structure.

In various embodiments, the initial structure is a 3-D hydrogel structure that is made of a hydrogel matrix. In various embodiments, the initial structure can include a wax, a plastic, or polyvinyl alcohol, PEGDA having 250-35,000 Da, PEG-norbornene, PEG-MMP, gelatin methacrylate, or any combination thereof. In various embodiments, one or more features comprises a vascular topology that is generated by additive or subtractive manufacturing. In various embodiments, the initial structure can be printed via a 3-D printer. In various embodiments, the initial structure can include a vascular topology within a tissue or organ.

In various embodiments, after the initial structure is printed, a post-process can be performed on the initial structure. In various embodiments, the post-process can include, among many others, washing the initial structure in a solvent, equilibrating the initial structure in a solvent, or crosslinking the initial structure in a lightbox.

In accordance with various embodiments, the method S400 includes, at step S404, casting a first material in one or more void volumes of the initial structure. In various embodiments, the first material can include, but not limited to, a thermoreversible material, metal or liquid metal including gallium, alloys including Field's metal, gallium-indium, gallium-tin, and gallium-indium-tin, carbohydrate glass, pluronic, low molecular weight PEG, PCL, gelatin, wax, or any combination thereof.

In various embodiments, the wax for the initial structure or the first material can be a wax from a diverse class of organic compounds. In some embodiments, the wax can be lipophilic and/or malleable solids near ambient temperatures. In various embodiments, the wax can include higher alkanes and lipids, with melting points above about 40° C. (104° F.), melting to give low viscosity liquids. In various embodiments, the wax can be insoluble in water but soluble in organic, nonpolar solvents.

In various embodiments, the wax can be an alkane hydrocarbon, where the alkane hydrocarbon has a formula CH₃(CH₂)ₙCH₃, where n=14-48. In various embodiments, the alkane hydrocarbon can be hexadecane, heptadecane, octadecane, eicosane, heneicosane, docosane, tetracosane, nonacosane, triacontane, hentriacontane, dotriacontane, hexatriacontane, tetracontane, tetratetracontane, or pentacontane. In various embodiments, the wax can be an alkene hydrocarbon, where the alkene hydrocarbon has a formula CH₃(CH₂)ₙCH═CH₂, where n=14-48. In various embodiments, the alkene hydrocarbon can be 1-octadecene.

In various embodiments, the wax can be an unsaturated hydrocarbon fatty alcohol. In various embodiments, the wax can be a saturated hydrocarbon fatty alcohol, where the saturated hydrocarbon can be decyl alcohol, lauryl alcohol, myristyl alcohol, palmityl alcohol, stearyl alcohol, arachidyl alcohol, behenyl alcohol, lignoceryl alcohol, ceryl alcohol, myricyl alcohol, or any combination thereof.

In various embodiments, the wax can be a lipid that contains saturated fatty acid, where the fatty acid has a formula CH₃(CH₂)ₙCOOH, where n=4-30. In various embodiments, the lipid can be hexanoic acid, nonanoic acid, octanoic acid, decanoic acid, methyl nonadecanoate, lauric acid, methyl arachidate, myristic acid, methyl tricosanoate, methyl behenate, heptadecanoic acid, palmitic acid, stearic acid, or melissic acid or any combination thereof.

In various embodiments, the wax can be a lipid that contains an unsaturated fatty acid, where the lipid is arachidonic acid, linolenic acid, palmitoleic acid, oleic acid, or any combinations thereof. In various embodiments, the wax can be a lipid combination that contains multiple saturated fatty acids or unsaturated fatty acids. In various embodiments, the wax can be a lipid combination that contains a saturated fatty acid or an unsaturated fatty acid.

In various embodiments, the wax is plant derived, animal derived, petroleum derived, or synthetic or any combination thereof.

In various embodiments, the wax can be paraffin, soy wax, beeswax (major component is myricyl palmitate), gulf wax, carnauba wax, candelilla wax, polyethylene wax, microcrystalline wax, or any combination thereof.

In various embodiments, the wax can contain additives, such as, for example but not limited to, stearic acid, glyceryl tristearate, sorbitan tristearate, beeswax, lecithin, or resins, such as, for example but not limited to, dammar gum, mastic gum, copal gum, shellac, to enhance mechanical properties.

In various embodiments, the wax can be diluted in peanut oil, sunflower oil, soybean oil canola oil, olive oil, or rice bran oil or any combination thereof.

In various embodiments, the wax can be prepared by transferring it into a container, heating them, then using them. In various embodiments, the wax can be melted at a higher temperature to use in an injection process at high temperature in an oven. In some embodiments, the wax can be processed at room temperature. In various embodiments, the wax can be combined with another wax to obtain desired physical, mechanical, and/or chemical properties. In some instances, more than one wax is combined to warm or to liquify before mixing them.

In various embodiments, the wax can be prepared using a casting process at a temperature range between about 4° C. and about 40° C. In various embodiments, the initial structure hydrogel can be incubated for solvent exchange for improved filling of the negative space by wax. In various embodiments, the hydrogel can be incubated in acetone or tetrahydrofuran or any combinations thereof. In various embodiments, the negative space of the initial structure is exposed to a lipophilic agent. In various embodiments, the lipophilic agent can be acetone or tetrahydrofuran.

In various embodiments, the cast structure contains a gallium or gallium containing material, such as, for example but not limited to, gallium-indium, gallium-tin, and gallium-indium-tin. In various embodiments, the cast structure has a surface roughness (Ra) less than 10 μm, 5 μm, 3 μm, 2 μm, 1 μm, 0.8 μm, 0.6 μm, 0.5 μm, 0.4 μm, 0.3 μm, 0.2 μm, or 0.1 μm. In various embodiments, the cast structure is post-processed by metallurgical techniques to provide additional features, including features with other surface properties. In various embodiments, where the cast structure includes independent vascular networks, the vascular networks can be post-processed independently. In various embodiments, where one network can be connected to an electrode and submerged in an electroplating bath and additional metal can be electroplated on top. In various embodiments, the electroplating is directionally applied and more at distal than proximal. In various embodiments, the electroplating is uniformly applied evenly over the surface. In various embodiments, the electroplating is the same metal or can be a different metal from the cast structure.

In various embodiments, dip coating can be used to modify the cast structure. In various embodiments, dip coating can be performed in sequential materials. In various embodiments, dip coating can be performed in solutions, which is then dried and solidified. In various embodiments, dip coating can be performed in solutions, which is then crystallized. In various embodiments, dip coating can be performed by nanometer-thick polyelectrolyte polymer films layer by layer. In various embodiments, dip coating can be performed in suspension of magnetic particles, magnetic field can align or control the surface changes. In various embodiments, the independent vascular networks are electrically charged and followed by powder casting. In various embodiments, the process includes laminin first, then follows by dip coating in collagen. In some embodiments, the process does not include a hard bake, such as required in those processes used in powder coating of parts for automobiles. In various embodiments, the cast structure can be coated using organic polymer in alternating layers (OPAL) process, where each coating layer can include a different material, which may give rise to a different color, along one or more directions on the surface and/or along the growth direction of the cast structure, which may form a series of colors (e.g., rainbow) across the surface. In other words, the cast structure can be coated to have a gradient in the coating by controlled mixing in the structure in both 2D and/or 3D arrangements of the coating, along a surface and/or through the layer of the coating.

In various embodiments, the gallium or gallium containing material, such as, for example but not limited to, gallium-indium, gallium-tin, and gallium-indium-tin, can be removed via electrophoresis. The remaining amount of the gallium content after removal can be determined via elemental analysis. In various embodiments, there may be less gallium content in the coating of the cast structure than before the coating is performed.

In various embodiments, the initial structure can include a hydrogel, where the hydrogel is alginate. In various embodiments, the alginate can be crosslinked with calcium chloride. In various embodiments, the alginate can be dissolved with a calcium chelator. In various embodiments, the chelator can be sodium citrate or ethylenediaminetetraacetic acid (EDTA).

In various embodiments, the initial structure can include hydrogel components. In various embodiments, the state of matter of the injected material can be altered by pH or salt concentration. In various embodiments, the injected material can involve formation of material through multivalent inorganic cations or anions. In various embodiments, the injected material can be a polysaccharide such as alginate, wherein the alginate can be crosslinked with calcium divalent cations from calcium chloride.

In various embodiments, the cast structure can be removed through the use of a cationic or anionic chelator. In various embodiments, the chemical used to dissolve the first material is a calcium chelator. In various embodiments, the chelator can be sodium citrate or EDTA. In various embodiments, the injected material can involve formation of material through disulfide bonds. In various embodiments, the cast structure can be removed under reducing conditions. In various embodiments, the injected material can involve a covalently adaptable network. In various embodiments, the adaptable bond can involve formation of a reversible thioester. In various embodiments, the injected material can involve a thermodynamically reversible network. In various embodiments, the thermodynamically reversible network can be formed by coupling of a furan and maleimide. In various embodiments, the thermodynamically reversible network can be initiated and reversed using heat/temperature. In various embodiments, the Diels Alder adduct product can be formed at 60° C. In various embodiments, the retro Diels Alder product can be formed at 110° C.

In various embodiments, the first material can be agarose. In various embodiments, the first material can be a biologically-derived polymeric material, wherein the material is gelatin. In various embodiments, the gelatin can be crosslinked by an enzymatic or chemical crosslinker. In various embodiments, the enzymatic crosslinker can be microbial transglutaminase. In various embodiments, the chemical crosslinker can be glutaraldehyde. In various embodiments, the gelatin cast can be removed by enzymatic degradation. In various embodiments, the enzyme can be collagenase.

In various embodiments, casting the first material in the one or more void volumes of the initial structure includes: filling the first material in the one or more void volumes of the initial structure at a first temperature; and solidifying the first material at a second temperature, wherein the first material at the first temperature and the first material at the second temperature have different physical states. In accordance with various embodiments, the first temperature for the first material, such as gallium and other liquid metals, ranges from about 65° C. to about 4° C., and for the first material, such as PCL, ranges from about 65° C. to about 35° C. In various embodiments, the second temperature for the first material, such as gallium and other liquid metals, and PCL, ranges from room temperature to about 4° C. In some embodiments, the cast material filling/solidification is not temperature sensitive and can be performed between about 37° C. and about 4° C.

In accordance with various embodiments, the method S400 includes, at step S406, removing the initial structure from the first material. In various embodiments, the initial structure can be removed via a number of processes and techniques, including for example, but not limited to, by dissolution or degradation, by one or more chemical processes under acidic or basic conditions, collagenase incubation with a protease or peptidase, including trypsin, collagenase, proteinase k, cathepsin K, or any combination thereof, or by one or more physical processes via a mechanical process, swelling, drying, heating, or via a light process due to presence of photolabile linkers in the initial structure, or any combination thereof. In various embodiments, metals that are liquid (e.g., liquid metal) at room or ambient temperature, but solid at lower temperatures, dissolution or degradation can be performed at low temperature, such as below 0° C. (273 Kelvin), by incubating the initial structure in a dissolution or degradation solution containing glycerol. In various embodiments, the cast structure can be removed by cooling. In various embodiments, the cast structure can be removed by adjusting the pH, for example, if alginate is used as the cast material; if PEGDA is used as the cast material (GelMA gel, PEGDA void cast, enzymatic dissolution or degradation, plastic cast, sodium hydroxide dissolution or degradation). In various embodiments, the cast structure can be removed by enzymes (trypsin, collagenase, etc.) if gelatin/GelMA material is used as the cast material (PEGDA gel, GelMA void cast, hydrolytic dissolution or degradation, PEG-norbornene cast, collagenase dissolution or degradation).

In accordance with various embodiments, the method S400 includes, at step S408, obtaining a cast structure comprising the first material. In various embodiments, the cast structure comprises a negative or inverted mold of the initial structure.

In accordance with various embodiments, the method S400 includes, at step S410, coating a second material on the cast structure. In various embodiments, the second material includes a biocompatible material, a hemocompatible material, cells, a cell-adhesive material, collagen, gelatin, fibronectin, laminin, polylysine, PEG based peptide conjugates, polyurethane, or any combination thereof, or a metal comprising copper, nickel, or gold.

In various embodiments, the second material is coated via dip-coating, spray coating, powder coating, vapor deposition, electroplating, or via oxidation and/or reduction reactions, or a combination thereof. In various embodiments, the second material is coated to a thickness between 10 nm and 1000 μm. In various embodiments, the one or more coatings can include a multilayer coating, wherein the multilayer coating is obtained by forming a coating at least two times. In various embodiments, the multilayer coating on the final structure comprises at least two different coating materials.

In accordance with various embodiments, the method S400 includes, at step S412, casting a third material using the coated cast structure. In various embodiments, the third material includes a biomaterial comprising silk, collagen, gelatin, fibrin, synthetic peptides, hyaluronic acid, polymers comprising alginate, polyurethane, polycaprolactone (PCL), elastomers, collagen methacrylate, collagen methacrylamide, gelatin methacrylate, gelatin methacrylamide, silk methacrylate, silk methacrylamide, hyaluronic acid methacrylate, hyaluronic acid methacrylamide, pluronic diacrylate, pluronic methacrylamide, chondroitin sulfate methacrylate, chondroitin sulfate methacrylamide, elastin methacrylate, elastin methacrylamide, cellulose acrylate, cellulose methacrylamide, dextran methacrylate, dextran methacrylamide, heparin methacrylate, heparin methacrylamide, N-isopropyl acrylamide (NIPAAm), chitosan methacrylate, chitosan methacrylamide, polyethylene glycol norbornene, polyethylene glycol dithiol, thiolated gelatin, thiolated chitosan, thiolated hyaluronic acid, thiolated silk, PEG based peptide conjugates, decellularized ECM of any tissue/organ, plastic, metal, including gallium or indium, or alloys, including Field's metal, gallium-indium, gallium-tin, and gallium-indium-tin, supercooled liquid metal, or any combination thereof.

In accordance with various embodiments, the method S400 includes, at step S414, removing the first material. In various embodiments, the cast structure is removed by dissolution or degradation, by one or more chemical processes under acidic or basic conditions, collagenase incubation with a protease or peptidase, including trypsin, collagenase, proteinase k, cathepsin K, or any combination thereof, or by one or more physical processes via a mechanical process, swelling, heating the cast structure to liquify the first material, or a light process due to presence of photolabile linkers in the initial structure, or any combination thereof. In various embodiments, metals that are liquid (e.g., liquid metal) at room or ambient temperature, but solid at lower temperatures, dissolution or degradation can be performed at low temperature, such as below 0° C. (273 Kelvin), by incubating the initial structure in a dissolution or degradation solution containing glycerol. In various embodiments, the cast structure can be removed by cooling. In various embodiments, the cast structure can be removed by adjusting the pH, for example, if alginate is used as the cast material; if PEGDA is used as the cast material (GelMA gel, PEGDA void cast, enzymatic dissolution or degradation, plastic cast, sodium hydroxide dissolution or degradation). In various embodiments, the cast structure can be removed by enzymes (trypsin, collagenase, etc.) if gelatin/GelMA material is used as the cast material (PEGDA gel, GelMA void cast, hydrolytic dissolution or degradation, PEG-norbornene cast, collagenase dissolution or degradation).

In accordance with various embodiments, the method S400 includes, at step S416, obtaining a final structure. In various embodiments, the final structure comprises the second material and the third material. In various embodiments, the final structure comprises the third material. In various embodiments, the final structure can be an artificial tissue or organ.

In accordance with various embodiments, the method S400 optionally includes, at step S418, modifying a surface of the cast structure prior to coating the second material. In various embodiments, modifying a surface of the cast structure further comprises roughening or smoothing of the surface, coating with a layer of collagen, coating a portion of the surface with collagen, electroplating, performing electrolysis, using ferro fluid in magnetic field to perform a surface treatment of the surface, or any combination thereof.

In various embodiments, the cast structure can be prepared for coating by lowering surface tension so that droplets do not form on the surface of the cast structure. In various embodiments, a coating material of the cast structure can contain a lipid. In various embodiments, the lipid can be a phospholipid, steroid, glycolipid, sphingolipid, or amphiphile or any combination thereof. In various embodiments, the phospholipid can be a zwitterionic phospholipid, or anionic phospholipid, or PEGylated phospholipid or any combination thereof. In various embodiments, the lipid can be polylactic acid. In various embodiments, the lipid can be a triglyceride. In various embodiments, the coating material can contain hydrophobic regions, wherein the hydrophobic containing material is a peptide or protein. In various embodiments, the material can be serum. In various embodiments, the hydrophobic material can be a lipid. In various embodiments, the coated structure allows for cell adhesion. In various embodiments, the cast structure is coated with a material to enable full removal of the first material in downstream applications, and the material is pluronic.

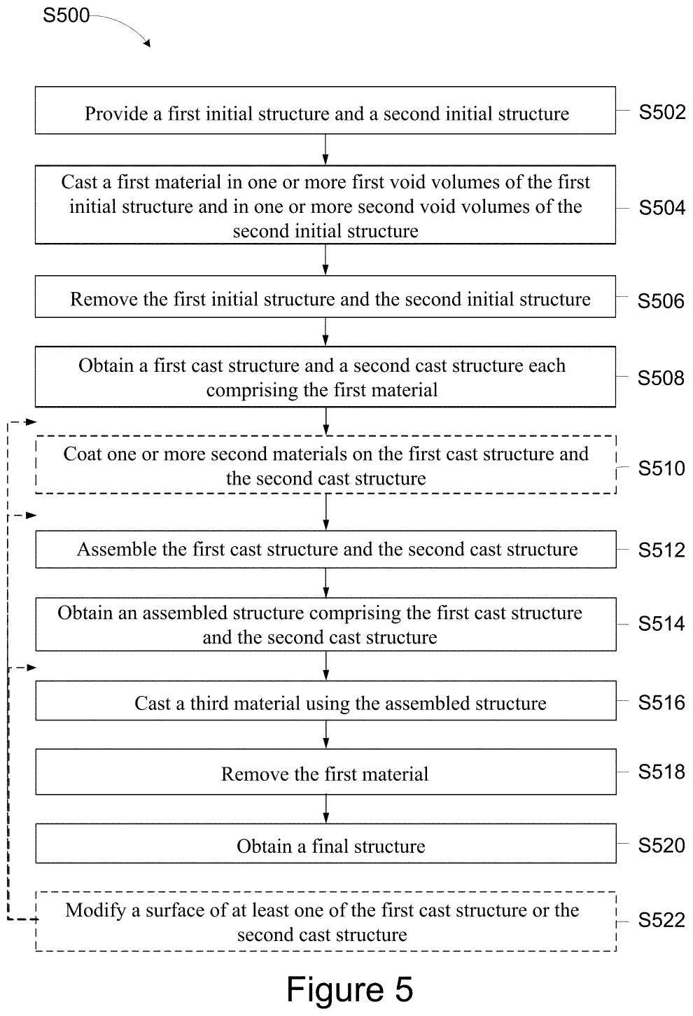

FIG. 5 is a flowchart for a method S500 for preparing a structure, according to various embodiments. In accordance with various embodiments, the method S500 includes, at step S502, providing a first initial structure and a second initial structure. In various embodiments, the first initial structure and/or the second initial structure can be printed and can have one or more features. In various embodiments, the one or more features of the first initial structure and the second initial structure can be positive or negative features. In accordance with various embodiments, the positive features refer to spaces that are occupied by a material that made up the first initial structure and/or the second initial structure. In accordance with various embodiments, the negative features refer to spaces that are absent of a material that made up the first initial structure and/or the second initial structure, e.g., a void space or a void volume within the first initial structure and/or the second initial structure. In accordance with various embodiments, the one or more negative features refer to one or more void volumes of the first initial structure and/or the second initial structure.

In various embodiments, at least one of the first initial structure or the second initial structure can be a 3-D hydrogel structure that is made of a hydrogel matrix. In various embodiments, at least one of the first initial structure or the second initial structure can include a wax, a plastic, or polyvinyl alcohol, PEGDA having 250-35,000 Da, PEG-norbornene, PEG-MMP, gelatin methacrylate, or any combination thereof. In various embodiments, one or more features comprises a vascular topology that is generated by additive or subtractive manufacturing. In various embodiments, the first initial structure and/or the second initial structure can be printed via a 3-D printer. In various embodiments, the first initial structure and/or the second initial structure can include a vascular topology within a tissue or organ. In various embodiments, the one or more first void volumes can form a first vascular topology and the one or more second void volumes can form a second vascular topology.

In various embodiments, after the first initial structure and/or the second initial structure are printed, a post-process can be performed on the first initial structure and/or the second initial structure. In various embodiments, the post-process can include, among many others, washing the first initial structure and/or the second initial structure in a solvent, equilibrating the first initial structure and/or the second initial structure in a solvent, or crosslinking the first initial structure and/or the second initial structure in a lightbox.

In accordance with various embodiments, the method S500 includes, at step S504, casting a first material in one or more first void volumes of the first initial structure and in one or more second void volumes of the second initial structure. In various embodiments, the first material can include, but not limited to, a thermoreversible material, metal or liquid metal including gallium, alloys including Field's metal, gallium-indium, gallium-tin, and gallium-indium-tin, carbohydrate glass, pluronic, low molecular weight PEG, PCL, gelatin, wax, or any combination thereof.

In various embodiments, the wax for at least one of the first initial structure or the second initial structure, and/or the first/cast material can be a wax from a diverse class of organic compounds. In some embodiments, the wax can be lipophilic and/or malleable solids near ambient temperatures. In various embodiments, the wax can include higher alkanes and lipids, with melting points above about 40° C. (104° F.), melting to give low viscosity liquids. In various embodiments, the wax can be insoluble in water but soluble in organic, nonpolar solvents.

In various embodiments, the wax can be an alkane hydrocarbon, where the alkane hydrocarbon has a formula CH₃(CH₂)ₙCH₃, where n=14-48. In various embodiments, the alkane hydrocarbon can be hexadecane, heptadecane, octadecane, eicosane, heneicosane, docosane, tetracosane, nonacosane, triacontane, hentriacontane, dotriacontane, hexatriacontane, tetracontane, tetratetracontane, or pentacontane. In various embodiments, the wax can be an alkene hydrocarbon, where the alkene hydrocarbon has a formula CH₃(CH₂)ₙCH═CH₂, where n=14-48. In various embodiments, the alkene hydrocarbon can be 1-octadecene.

In various embodiments, the wax can be an unsaturated hydrocarbon fatty alcohol. In various embodiments, the wax can be a saturated hydrocarbon fatty alcohol, where the saturated hydrocarbon can be decyl alcohol, lauryl alcohol, myristyl alcohol, palmityl alcohol, stearyl alcohol, arachidyl alcohol, behenyl alcohol, lignoceryl alcohol, ceryl alcohol, myricyl alcohol, or any combination thereof.

In various embodiments, the wax can be a lipid that contains saturated fatty acid, where the fatty acid has a formula CH₃(CH₂)ₙCOOH, where n=4-30. In various embodiments, the lipid can be hexanoic acid, nonanoic acid, octanoic acid, decanoic acid, methyl nonadecanoate, lauric acid, methyl arachidate, myristic acid, methyl tricosanoate, methyl behenate, heptadecanoic acid, palmitic acid, stearic acid, or melissic acid or any combination thereof.

In various embodiments, the wax can be a lipid that contains an unsaturated fatty acid, where the lipid is arachidonic acid, linolenic acid, palmitoleic acid, oleic acid, or any combinations thereof. In various embodiments, the wax can be a lipid combination that contains multiple saturated fatty acids or unsaturated fatty acids. In various embodiments, the wax can be a lipid combination that contains a saturated fatty acid or an unsaturated fatty acid.

In various embodiments, the wax is plant derived, animal derived, petroleum derived, or synthetic or any combination thereof.

In various embodiments, the wax can be paraffin, soy wax, beeswax (major component is myricyl palmitate), gulf wax, carnauba wax, candelilla wax, polyethylene wax, microcrystalline wax, or any combination thereof.

In various embodiments, the wax can contain additives, such as, for example but not limited to, stearic acid, glyceryl tristearate, sorbitan tristearate, beeswax, lecithin, or resins, such as, for example but not limited to, dammar gum, mastic gum, copal gum, shellac, to enhance mechanical properties.

In various embodiments, the wax can be diluted in peanut oil, sunflower oil, soybean oil canola oil, olive oil, or rice bran oil or any combination thereof.

In various embodiments, the wax can be prepared by transferring it into a container, heating them, then using them. In various embodiments, the wax can be melted at a higher temperature to use in an injection process at high temperature in an oven. In some embodiments, the wax can be processed at room temperature. In various embodiments, the wax can be combined with another wax to obtain desired physical, mechanical, and/or chemical properties. In some instances, more than one wax is combined to warm or to liquify before mixing them.

In various embodiments, the wax can be prepared using a casting process at a temperature range between about 4° C. and about 40° C. In various embodiments, the initial structure hydrogel can be incubated for solvent exchange for improved filling of the negative space by wax. In various embodiments, the hydrogel can be incubated in acetone or tetrahydrofuran or any combinations thereof. In various embodiments, the negative space of the initial structure is exposed to a lipophilic agent. In various embodiments, the lipophilic agent can be acetone or tetrahydrofuran.

In various embodiments, the cast structure contains a gallium or gallium containing material, such as, for example but not limited to, gallium-indium, gallium-tin, and gallium-indium-tin. In various embodiments, the cast structure has a surface roughness (Ra) less than 10 μm, 5 μm, 3 μm, 2 μm, 1 μm, 0.8 μm, 0.6 μm, 0.5 μm, 0.4 μm, 0.3 μm, 0.2 μm, or 0.1 μm. In various embodiments, the cast structure is post-processed by metallurgical techniques to provide additional features, including features with other surface properties. In various embodiments, where the cast structure includes independent vascular networks, the vascular networks can be post-processed independently. In various embodiments, where one network can be connected to an electrode and submerged in an electroplating bath and additional metal can be electroplated on top. In various embodiments, the electroplating is directionally applied and more at distal than proximal. In various embodiments, the electroplating is uniformly applied evenly over the surface. In various embodiments, the electroplating is the same metal or can be a different metal from the cast structure.

In various embodiments, dip coating can be used to modify the cast structure. In various embodiments, dip coating can be performed in sequential materials. In various embodiments, dip coating can be performed in solutions, which is then dried and solidified. In various embodiments, dip coating can be performed in solutions, which is then crystallized. In various embodiments, dip coating can be performed by nanometer-thick polyelectrolyte polymer films layer by layer. In various embodiments, dip coating can be performed in suspension of magnetic particles, magnetic field can align or control the surface changes. In various embodiments, the independent vascular networks are electrically charged and followed by powder casting. In various embodiments, the process includes laminin first, then follows by dip coating in collagen. In some embodiments, the process does not include a hard bake, such as required in those processes used in powder coating of parts for automobiles. In various embodiments, the cast structure can be coated using organic polymer in alternating layers (OPAL) process, where each coating layer can include a different material, which may give rise to a different color, along one or more directions on the surface and/or along the growth direction of the cast structure, which may form a series of colors (e.g., rainbow) across the surface. In other words, the cast structure can be coated to have a gradient in the coating by controlled mixing in the structure in both 2D and/or 3D arrangements of the coating, along a surface and/or through the layer of the coating.

In various embodiments, the gallium or gallium containing material, such as, for example but not limited to, gallium-indium, gallium-tin, and gallium-indium-tin, can be removed via electrophoresis. The remaining amount of the gallium content after removal can be determined via elemental analysis. In various embodiments, there may be less gallium content in the coating of the cast structure than before the coating is performed.

In various embodiments, the initial structure can include a hydrogel, where the hydrogel is alginate. In various embodiments, the alginate can be crosslinked with calcium chloride. In various embodiments, the alginate can be dissolved with a calcium chelator. In various embodiments, the chelator can be sodium citrate or ethylenediaminetetraacetic acid (EDTA).

In various embodiments, the initial structure can include hydrogel components. In various embodiments, the state of matter of the injected material can be altered by pH or salt concentration. In various embodiments, the injected material can involve formation of material through multivalent inorganic cations or anions. In various embodiments, the injected material can be a polysaccharide such as alginate, wherein the alginate can be crosslinked with calcium divalent cations from calcium chloride.

In various embodiments, the cast structure can be removed through the use of a cationic or anionic chelator. In various embodiments, the chemical used to dissolve the first material is a calcium chelator. In various embodiments, the chelator can be sodium citrate or EDTA. In various embodiments, the injected material can involve formation of material through disulfide bonds. In various embodiments, the cast structure can be removed under reducing conditions. In various embodiments, the injected material can involve a covalently adaptable network. In various embodiments, the adaptable bond can involve formation of a reversible thioester. In various embodiments, the injected material can involve a thermodynamically reversible network. In various embodiments, the thermodynamically reversible network can be formed by coupling of a furan and maleimide. In various embodiments, the thermodynamically reversible network can be initiated and reversed using heat/temperature. In various embodiments, the Diels Alder adduct product can be formed at 60° C. In various embodiments, the retro Diels Alder product can be formed at 110° C.

In various embodiments, the first material can be agarose. In various embodiments, the first material can be a biologically-derived polymeric material, wherein the material is gelatin. In various embodiments, the gelatin can be crosslinked by an enzymatic or chemical crosslinker. In various embodiments, the enzymatic crosslinker can be microbial transglutaminase. In various embodiments, the chemical crosslinker can be glutaraldehyde. In various embodiments, the gelatin cast can be removed by enzymatic degradation. In various embodiments, the enzyme can be collagenase.

In various embodiments, casting the first material in the one or more first void volumes and in the one or more second void volumes comprises: filling the first material at a first temperature; and solidifying the first material at a second temperature, wherein the first material at the first temperature and the first material at the second temperature have different physical states.

In accordance with various embodiments, the method S500 includes, at step S506, removing the first initial structure and the second initial structure. In various embodiments, at least one of the first initial structure or the second initial structure can be removed via a number of processes and techniques, including for example, but not limited to, by dissolution or degradation, by one or more chemical processes under acidic or basic conditions, collagenase incubation with a protease or peptidase, including trypsin, collagenase, proteinase k, cathepsin K, or any combination thereof, or by one or more physical processes via a mechanical process, swelling, drying, heating, or via a light process due to presence of photolabile linkers in the initial structure, or any combination thereof. In various embodiments, metals that are liquid (e.g., liquid metal) at room or ambient temperature, but solid at lower temperatures, dissolution or degradation can be performed at low temperature, such as below 0° C. (273 Kelvin), by incubating the initial structure in a dissolution or degradation solution containing glycerol.

In accordance with various embodiments, the method S500 includes, at step S508, obtaining a first cast structure and a second cast structure each comprising the first material. In various embodiments, the first cast structure comprises a negative or inverted mold of the first initial structure. In various embodiments, the second cast structure comprises a negative or inverted mold of the second initial structure.

In accordance with various embodiments, the method S500 optionally includes, at step S510, coating one or more second materials on the first cast structure and the second cast structure. In various embodiments, the one or more second materials can include a biocompatible material, a hemocompatible material, cells, a cell-adhesive material, collagen, gelatin, fibronectin, laminin, polylysine, PEG based peptide conjugates, polyurethane, or any combination thereof, or a metal comprising copper, nickel, or gold.

In various embodiments, the one or more second materials are coated on at least one of the first cast structure or the second cast structure via dip-coating, spray coating, powder coating, vapor deposition, electroplating, or via oxidation and/or reduction reactions, or a combination thereof. In various embodiments, the one or more second materials are coated on at least one of the first cast structure or the second cast structure to a thickness between 10 nm and 1000 μm.

In various embodiments, the one or more second materials are coated on at least one of the first cast structure or the second cast structure at least two times to form a multilayer coating. In various embodiments, the multilayer coating on the at least one of the first cast structure or the second cast structure can include at least two different coating materials.

In various embodiments, the one or more second materials that is coated on the first cast structure has a different thickness than the second material that is coated on the second cast structure. In various embodiments, the one or more second materials that is coated on the first cast structure has a different surface roughness value than the second material that is coated on the second cast structure. In various embodiments, the first cast structure may have different surface roughness values on different parts of the first cast structure. In various embodiments, the second cast structure may have different surface roughness values on different parts of the second cast structure. In various embodiments, the first cast structure or the second cast structure may have different surface roughness values at desired locations on the respective cast structure. In various embodiments, the one or more second materials that is coated on the first cast structure is different from the one or more second materials that is coated on the second cast structure. In various embodiments, nickel is coated on the first cast structure and gold is coated on the second cast structure, or vice versa.

In accordance with various embodiments, the method S500 includes, at step S512, assembling the first cast structure and the second cast structure.

In accordance with various embodiments, the method S500 includes, at step S514, obtaining an assembled structure comprising the first cast structure and the second cast structure. In various embodiments, the assembled structure can include one or more first features of the first cast structure and one or more second features of the second cast structure. In various embodiments, the assembled structure is a capacitor.

In accordance with various embodiments, the method S500 includes, at step S516, casting a third material using the assembled structure. In various embodiments, the third material comprises a biomaterial comprising silk, collagen, gelatin, fibrin, synthetic peptides, hyaluronic acid, polymers comprising alginate, polyurethane, polycaprolactone (PCL), elastomers, collagen methacrylate, collagen methacrylamide, gelatin methacrylate, gelatin methacrylamide, silk methacrylate, silk methacrylamide, hyaluronic acid methacrylate, hyaluronic acid methacrylamide, pluronic diacrylate, pluronic methacrylamide, chondroitin sulfate methacrylate, chondroitin sulfate methacrylamide, elastin methacrylate, elastin methacrylamide, cellulose acrylate, cellulose methacrylamide, dextran methacrylate, dextran methacrylamide, heparin methacrylate, heparin methacrylamide, N-isopropyl acrylamide (NIPAAm), chitosan methacrylate, chitosan methacrylamide, polyethylene glycol norbornene, polyethylene glycol dithiol, thiolated gelatin, thiolated chitosan, thiolated hyaluronic acid, thiolated silk, PEG based peptide conjugates, decellularized ECM of any tissue/organ, plastic, metal, including gallium or indium, or alloys, including Field's metal, gallium-indium, gallium-tin, and gallium-indium-tin, supercooled liquid metal, or any combination thereof.

In various embodiments, the method can further include heating to expand the one or more first features or the one or more second features prior to casting the third material using the assembled structure at step S516. Similarly, in various embodiments, the method can further include cooling to shrink the one or more first features or the one or more second features prior to casting the third material using the assembled structure.

In accordance with various embodiments, the method S500 includes, at step S518, removing the first material. In various embodiments, the first material is removed by heating to liquify the first material in one of the first cast structure or the second cast structure. In various embodiments, the first material is removed by dissolution or degradation, by one or more chemical processes under acidic or basic conditions, collagenase incubation with a protease or peptidase, including trypsin, collagenase, proteinase k, cathepsin K, or any combination thereof, or by one or more physical processes via a mechanical process, swelling, drying, heating the first cast structure or the second cast structure to liquify the first material, or a light process due to presence of photolabile linkers in the initial structure, or any combination thereof. In various embodiments, metals that are liquid (e.g., liquid metal) at room or ambient temperature, but solid at lower temperatures, dissolution or degradation can be performed at low temperature, such as below 0° C. (273 Kelvin), by incubating the initial structure in a dissolution or degradation solution containing glycerol.

In accordance with various embodiments, the method S500 includes, at step S520, obtaining a final structure. In various embodiments, the final structure comprises the one or more second materials and the third material. In various embodiments, the final structure comprises the third material. In various embodiments, the final structure can be an artificial tissue or organ.

In accordance with various embodiments, the method S500 optionally includes, at step S522, modifying a surface of at least one of the first cast structure or the second cast structure prior to coating the one or more second materials at optional step S510, prior to assembling at step S512, or after assembling (obtained the assembled structure) at step S514. In various embodiments, modifying the surface can include roughening or smoothing of the surface, coating with a layer of collagen, coating a portion of the surface with collagen, electroplating, performing electrolysis, using ferro fluid in magnetic field to perform a surface treatment of the surface, or any combination thereof.

In various embodiments, the cast structure can be prepared for coating by lowering surface tension so that droplets do not form on the surface of the cast structure. In various embodiments, a coating material of the cast structure can contain a lipid. In various embodiments, the lipid can be a phospholipid, steroid, glycolipid, sphingolipid, or amphiphile or any combination thereof. In various embodiments, the phospholipid can be a zwitterionic phospholipid, or anionic phospholipid, or PEGylated phospholipid or any combination thereof. In various embodiments, the lipid can be polylactic acid. In various embodiments, the lipid can be a triglyceride. In various embodiments, the coating material can contain hydrophobic regions, wherein the hydrophobic containing material is a peptide or protein. In various embodiments, the material can be serum. In various embodiments, the hydrophobic material can be a lipid. In various embodiments, the coated structure allows for cell adhesion. In various embodiments, the cast structure is coated with a material to enable full removal of the first material in downstream applications, and the material is pluronic.

In various embodiments, the one or more first features and the one or more second features are separated by a distance of larger than 1 μm at a nearest point of separation between the one or more first features and the one or more second features. In various embodiments, the final structure comprises one or more final first features that is a negative mold of the one or more first features of the first cast structure and one or more final second features that is a negative mold of the one or more second features of the second cast structure. In various embodiments, the one or more final first features and the one or more final second features are separated by a distance of less than 10 μm at a nearest point of separation between the one or more final first features and the one or more final second features.

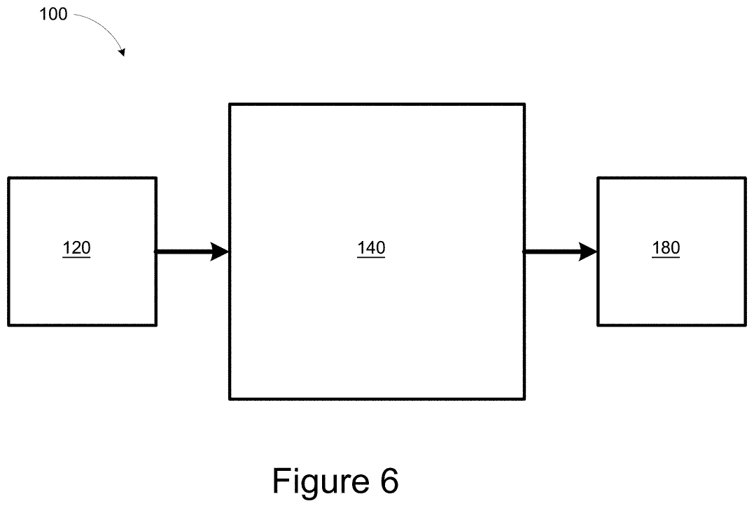

FIG. 6 is a block diagram that illustrates a process 100 of transforming an initial structure 120 to a final structure 180, according to various embodiments. FIG. 6 illustrates the initial structure 120 that is used to process via a method 140 to obtain the final structure 180. The method 140 can be any of the methods S100, S200, S300, S400, or S500 as described with respect to FIGS. 1-5.

In accordance with various embodiments, a structure can be produced according to any of the methods S100, S200, S300, S400, or S500 as described with respect to FIGS. 1-5. In various embodiments, the structure can include a fluid channel for casting one or more of the first and second materials, similar to the materials as described herein. In various embodiments, the fluid channel is within a void volume of the initial structure. In various embodiments, the fluid channel comprises one or more constrictions to regulate a flow of injectable material. In various embodiments, one or more constrictions is designed as a terminal feature. In various embodiments, one or more constrictions is configured as a valve to terminate a void volume after filling the injectable material.

In accordance with various embodiments, a system for producing a structure according to any of the methods S100, S200, S300, S400, or S500 as described with respect to FIGS. 1-5. In various embodiments, the system includes an initial structure comprising a hydrogel, a chamber to house the hydrogel, a manifold to connect to hydrogel vessel inlets and outlets, one or more tubing connected to the manifold, one or more pumps, one or more fluid reservoirs, and/or one or more waste containers. In various embodiments, one or more pumps comprises a syringe pump or a peristaltic pump. In various embodiments, one or more pumps are used to fill an injectable material into one or more voids within the initial structure. In various embodiments, one or more voids within the initial structure are filled using a positive or negative pressure. In various embodiments, an electrical gradient is applied to fill the one or more voids within the initial structure. In various embodiments, a vacuum is applied to fill the one or more voids within the initial structure.

### EXAMPLES

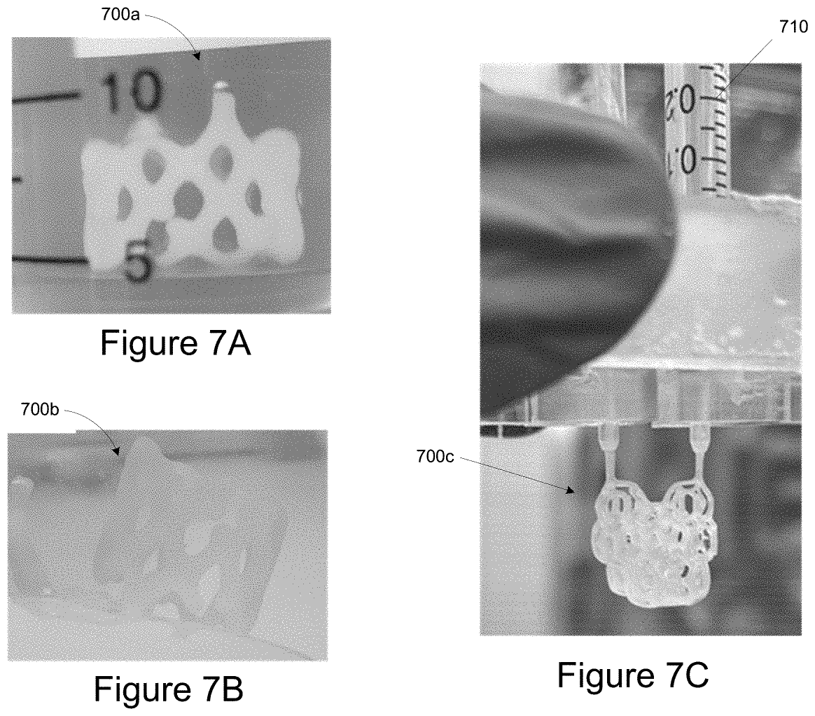

FIGS. 7-13 are images of finished samples and sample structures at various stages of the structure preparation using one or more of the methods S100, S200, S300, S400, or S500 as described with respect to FIGS. 1-5.

FIG. 7A shows a sample structure 700a prepared using wax, in accordance with various embodiments. As shown in FIG. 7A, the sample structure 700a comprises a hydrogel, composed of polyethylene glycol diacrylate that contains an empty channel, which was 3D printed. The 3D-printed channel of the sample structure 700a is then injected with pre-warmed Ghee butter. The sample structure 700a shown in FIG. 7A is an injected Ghee butter in the voids of the printed hydrogel.

FIG. 7B shows a sample structure 700b obtained after the Ghee butter solidified within the 3D-printed hydrogel channel of the sample structure 700a, in accordance with various embodiments. To obtain the sample structure 700b, the hydrogel sample structure 700a is placed in a solution containing either an acid or a base to enable the PEGDA hydrogel to degrade. Once the hydrogel is fully degraded, the Ghee butter vascular cast remains and results in the sample structure 700b. As shown in FIG. 7B, the sample structure 700b is the Ghee butter vascular cast, which is obtained after hydrogel dissolution or degradation in an aqueous solution of the sample structure 700a shown in FIG. 7A.

FIG. 7C shows an example structure 700c, which is a more complicated and/or convoluted structure having 3D vessels, in accordance with various embodiments. To obtain the example structure 700c shown in FIG. 7C, an initial structure having the hydrogel is 3D printed, along with a 2-part manifold. A top part (shown here) of the 2-part manifold is designed to allow fluidic connections to the hydrogel and a chamber (not shown) of the 2-part manifold is designed to house the hydrogel during a wax injection and hydrogel dissolution or degradation process. A syringe 710 is connected to the top part, and pre-warmed wax is injected through the top part, which is then flowed into the hydrogel channel and out into a collection container. The wax is then allowed to solidify, then acid or base is added to the chamber to degrade the hydrogel. Once the hydrogel is degraded, the top manifold part is lifted from the bottom part, to reveal a wax vascular cast, which is the sample structure 700c shown in FIG. 7C. In accordance with various embodiments, the top part is configured to be in fluid communication with the connected cast, which can be moved into another reservoir for post processing. The post processing may include surface coating of the cast or polymerization of another material surrounding the cast, with possibly removing the casted wax material from the final object.

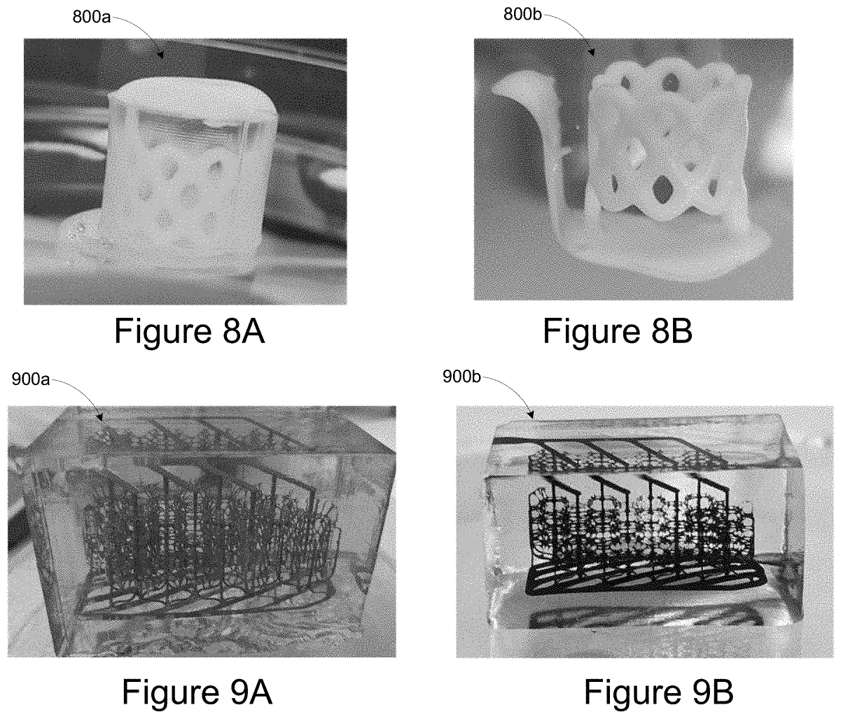

FIG. 8A shows a 3D printed hydrogel 800a composed of polyethylene glycol diacrylate (PEGDA, in yellow) which contained an empty vascular network inside, in accordance with various embodiments. To produce this sample, the hydrogel is injected with a warm 60% polycaprolactone (PCL) solution (dissolved in DMSO) to fill the empty voids within the hydrogel. Then the PCL is allowed to solidify at 4° C.

FIG. 8B shows an example structure 800b after the PCL solidified within the hydrogel, in accordance with various embodiments. The hydrogel is placed in a solution containing either acid or base to enable the PEGDA hydrogel to degrade. Once the hydrogel is fully degraded, the PCL vascular cast remains, resulting in the example structure 800b, as shown in FIG. 8B.

FIG. 9A shows an example structure 900a comprising a 3D printed PEG hydrogel injected with a red dye (India Ink colloid), which is produced in accordance with various embodiments.

FIG. 9B shows an example structure 900b comprising a 3D printed PEG hydrogel injected with a blue colloid, which is produced in accordance with various embodiments.

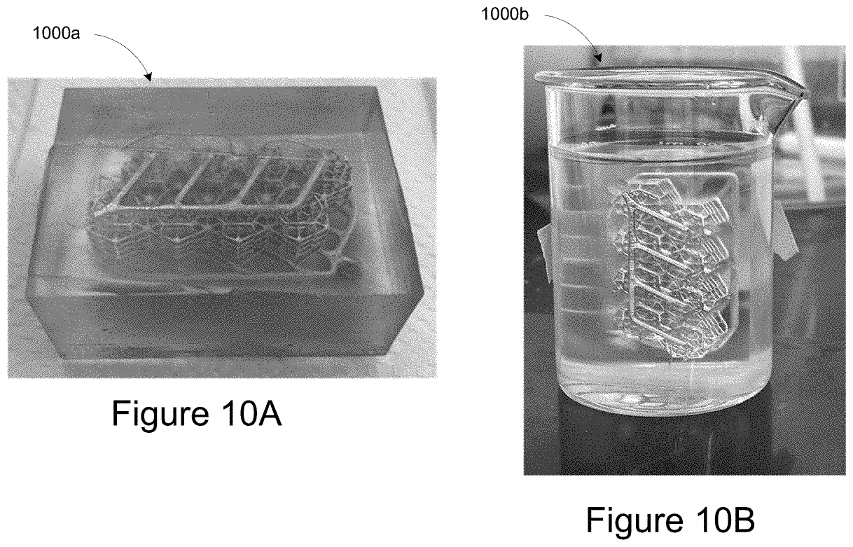

FIG. 10A shows an example structure 1000a comprising a 3D printed resin, in accordance with various embodiments. The sample structure 1000b as shown in FIG. 10A is obtained by filling the 3D printed resin with liquid metal Ga.

FIG. 10B shows an example structure 1000b which results from a 3D printed resin being dissolved in a suitable solution, such as a base solution up to 10 N NaOH. The 3D printed resin is shown being dissolved inside a beaker containing the base solution.

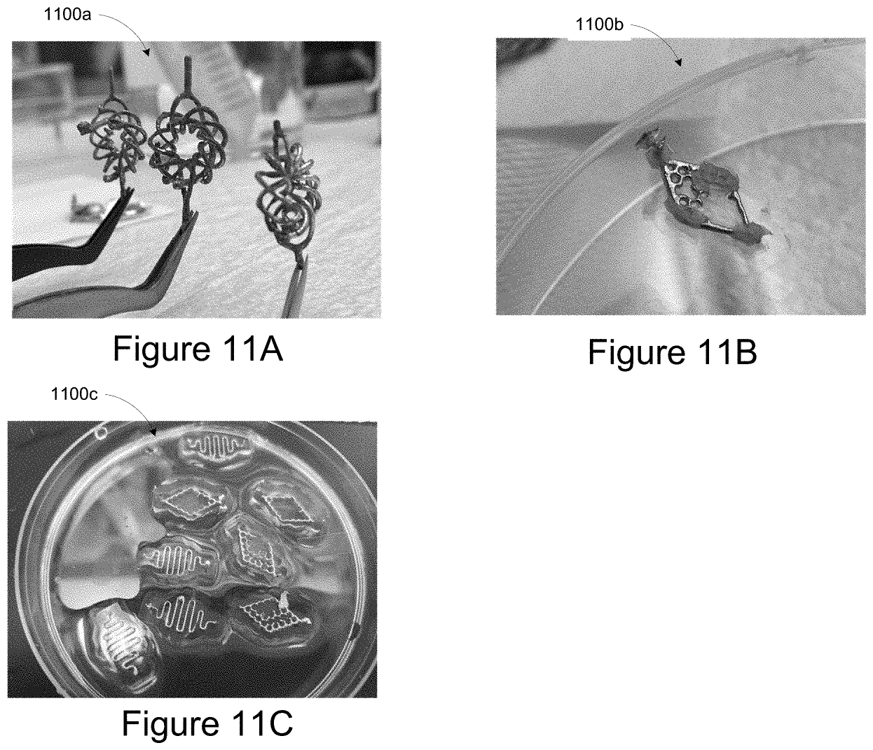

FIG. 11A shows example structures 1100a which are released cast structures (multivascular), in accordance with various embodiments. The example structures 1100a are torous knotts and comprise 20 wt % PEG hydrogel, which are obtained by releasing using a solution containing, for example, 0.1 N NaOH.

FIG. 11B shows an example structure 1100b, in accordance with various embodiments. As shown in FIG. 11B, the example structure 1100b is a Ga solidified structure that is casted in hyrogel with a partially dehydrated hydrogel.

FIG. 11C shows multiple example structures 1100c, which are printed hydrogels (in yellow), in accordance with various embodiments. The example structures 1100c shown in FIG. 11C are then injected with Ga into the empty channel. The Ga is then allowed to solidify to illustrate incomplete filling of some vessels with Ga in the channel, which is apparent when the hydrogel is dissolved.

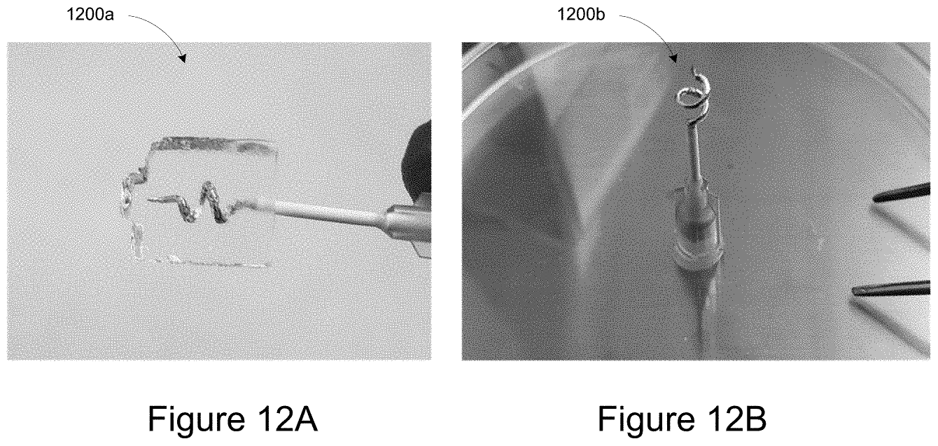

FIG. 12A shows a printed hydrogel structure 1200a, in accordance with various embodiments. The hydrogel structure 1200a shown in FIG. 12A includes an empty spiral channel which is filled with Ga.

FIG. 12B shows a cast structure 1200b, in accordance with various embodiments. The cast structure 1200b shown in FIG. 12B is obtained after the hydrogel is degraded, for example, via a suitable acid or base solution. As shown in the figure, the cast structure 1200b is a Ga cast structure remained attached to the fluidic tip.

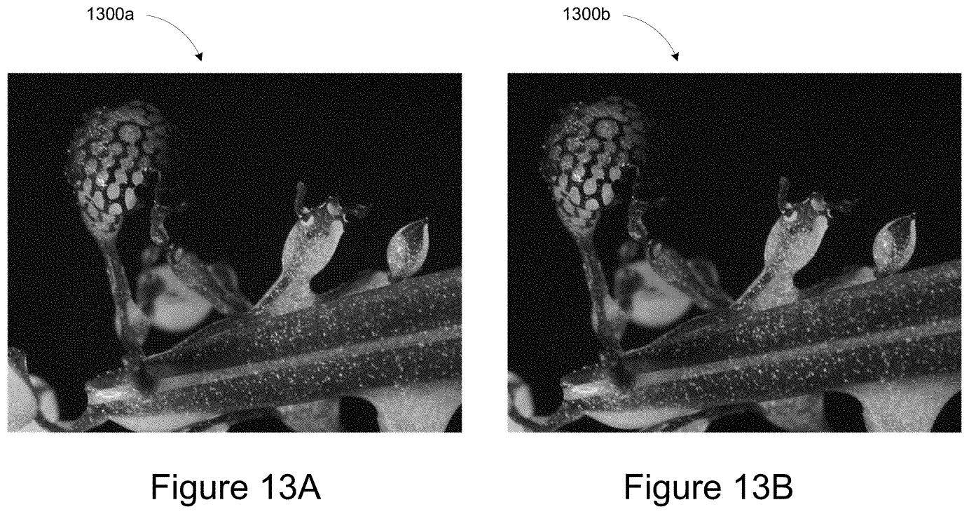

FIG. 13A shows a cast vascular structure 1300a, in accordance with various embodiments. The released cast vascular structure 1300a shown in FIG. 13A is sequentially coated in silicone with green fluorescent beads, followed by red fluorescent beads.

FIG. 13B shows a cast vascular structure 1300b, in accordance with various embodiments. The released cast vascular structure 1300b shown in FIG. 13B is sequentially coated in silicone with red fluorescent beads, followed by green fluorescent beads.

### RECITATION OF EMBODIMENTS

Embodiment 1: A method of preparing a structure, comprising: providing an initial structure having one or more features; performing a post-process of the initial structure; casting a material using the post-processed initial structure; removing the initial structure from the cast material; and obtaining a final structure comprising the cast material.

Embodiment 2. The method of Embodiment 1, wherein the initial structure is a 3-D hydrogel structure comprising a hydrogel matrix.

Embodiment 3. The method of any preceding Embodiment, wherein the initial structure comprises a wax, a plastic, or polyvinyl alcohol, polyethylene glycol diacrylate (PEGDA) having 250-35,000 Da, PEG-norbornene, polyethylene glycol diacrylamide (PEGDAAm) having 250-35,000 Da, MMP-sensitive PEGs (PEG-MMP), gelatin methacrylate, or any combination thereof.

Embodiment 4. The method of any preceding Embodiment, wherein the one or more features are generated by additive or subtractive manufacturing.

Embodiment 5. The method of any preceding Embodiment, wherein the post-process comprises washing the initial structure in a solvent, equilibrating the initial structure in a solvent, or crosslinking the initial structure in a lightbox.

Embodiment 6. The method of any preceding Embodiment, wherein the cast material comprises a biomaterial comprising silk, collagen, gelatin, fibrin, synthetic peptides, hyaluronic acid, polymers comprising alginate, polyurethane, polycaprolactone (PCL), elastomers, collagen methacrylate, collagen methacrylamide, gelatin methacrylate, gelatin methacrylamide, silk methacrylate, silk methacrylamide, hyaluronic acid methacrylate, hyaluronic acid methacrylamide, pluronic diacrylate, pluronic methacrylamide, chondroitin sulfate methacrylate, chondroitin sulfate methacrylamide, elastin methacrylate, elastin methacrylamide, cellulose acrylate, cellulose methacrylamide, dextran methacrylate, dextran methacrylamide, heparin methacrylate, heparin methacrylamide, N-isopropyl acrylamide (NIPAAm), chitosan methacrylate, chitosan methacrylamide, polyethylene glycol norbornene, polyethylene glycol dithiol, thiolated gelatin, thiolated chitosan, thiolated hyaluronic acid, thiolated silk, PEG based peptide conjugates, decellularized ECM of any tissue/organ, plastic, metal, including gallium or indium, or alloys, including Field's metal, gallium-indium, gallium-tin, and gallium-indium-tin, supercooled liquid metal, or any combination thereof.

Embodiment 7. The method of any preceding Embodiment, wherein the initial structure is removed by dissolution or degradation, by one or more chemical processes under acidic or basic conditions, collagenase incubation with a protease or peptidase, including trypsin, collagenase, proteinase k, cathepsin K, or any combination thereof, or by one or more physical processes via a mechanical process, swelling, drying, heating, or via a light process due to presence of photolabile linkers in the initial structure, or any combination thereof.

Embodiment 8. The method of any preceding Embodiment, wherein the initial structure comprises a vascular topology within a tissue or organ.

Embodiment 9. The method of any preceding Embodiment, wherein the final structure is an artificial tissue or organ, a tissue model, a phantom, or a stent.

Embodiment 10. The method of any preceding Embodiment, further comprising: modifying a surface of the final structure.

Embodiment 11. The method of Embodiment 10, wherein modifying the surface of the final structure further comprises roughening or smoothing of the surface, coating with a layer of collagen, coating a portion of the surface with collagen, electroplating, performing electrolysis, using ferro fluid in magnetic field to perform a surface treatment of the surface, or any combination thereof.

Embodiment 12. The method of any preceding Embodiment, further comprising: forming one or more coatings on the final structure.

Embodiment 13. The method of Embodiment 12, wherein the one or more coatings comprises a biocompatible material, a hemocompatible material, cells, a cell-adhesive material, collagen, gelatin, fibronectin, laminin, polylysine, PEG based peptide conjugates, polyurethane, or any combination thereof, or a metal comprising copper, nickel, or gold.

Embodiment 14. The method of any preceding Embodiment, wherein the one or more coatings has a thickness between 10 nm and 1000 μm.

Embodiment 15. The method of any preceding Embodiment, wherein the one or more coatings comprises a multilayer coating, wherein the multilayer coating is obtained by forming a coating at least two times.

Embodiment 16. The method of Embodiment 15, wherein the multilayer coating on the final structure comprises at least two different coating materials.

Embodiment 17. The method of any preceding Embodiment, further comprising: performing a post-process of the final structure.

Embodiment 18. The method of Embodiment 17, wherein the final structure is equilibrated in solvent.

Embodiment 19. The method of any preceding Embodiment, wherein the final structure is crosslinked in a lightbox.

Embodiment 20. A method of preparing a structure, comprising: providing an initial structure; casting a first material in one or more void volumes of the initial structure; removing the initial structure from the first material; obtaining a cast structure comprising the first material; casting a second material using the cast structure; removing the cast structure from the second material; and obtaining a final structure comprising the second material.

Embodiment 21. The method of Embodiment 20, wherein the initial structure is a hydrogel matrix structure generated by additive or subtractive manufacturing.

Embodiment 22. The method of any one of Embodiments 20 or 21, wherein the initial structure comprises a wax, a plastic, or polyvinyl alcohol, polyethylene glycol diacrylate (PEGDA) having 250-35,000 Da, PEG-norbornene, MMP-sensitive PEGs (PEG-MMP), gelatin methacrylate, or any combination thereof.

Embodiment 23. The method of any one of Embodiments 20-22, wherein the one or more void volumes forms a vascular topology.

Embodiment 24. The method of any one of Embodiments 20-23, wherein the initial structure is post-processed via washing or equilibrating in a solvent, or by crosslinking in a lightbox.

Embodiment 25. The method of any one of Embodiments 20-24, wherein the first material comprises a thermoreversible material, metal or liquid metal including gallium, alloys including Field's metal, gallium-indium, gallium-tin, and gallium-indium-tin, supercooled liquid metal, carbohydrate glass, pluronic, low molecular weight PEG, PCL, gelatin, wax, or any combination thereof.

Embodiment 26. The method of any one of Embodiments 20-25, wherein casting the first material in the one or more void volumes of the initial structure comprises: filling the first material in the one or more void volumes of the initial structure at a first temperature; and solidifying the first material at a second temperature, wherein the first material at the first temperature and the first material at the second temperature have different physical states.

Embodiment 27. The method of any one of Embodiments 20-26, wherein casting the first material in the one or more void volumes of the initial structure comprises: injecting the first material in the one or more void volumes of the initial structure at a first temperature; supercooling the first material; and crystalizing the first material at a second temperature.

Embodiment 28. The method of any one of Embodiments 20-27, wherein the initial structure is removed by dissolution or degradation, by one or more chemical processes under acidic or basic conditions, collagenase incubation with a protease or peptidase, including trypsin, collagenase, proteinase k, cathepsin K, or any combination thereof, or by one or more physical processes via a mechanical process, swelling, drying, heating, or via a light process due to presence of photolabile linkers in the initial structure, or any combination thereof.

Embodiment 29. The method of any one of Embodiments 20-28, wherein the second material comprises a biomaterial comprising silk, collagen, gelatin, fibrin, synthetic peptides, hyaluronic acid, polymers comprising alginate, polyurethane, polycaprolactone (PCL), elastomers, collagen methacrylate, collagen methacrylamide, gelatin methacrylate, gelatin methacrylamide, silk methacrylate, silk methacrylamide, hyaluronic acid methacrylate, hyaluronic acid methacrylamide, pluronic diacrylate, pluronic methacrylamide, chondroitin sulfate methacrylate, chondroitin sulfate methacrylamide, elastin methacrylate, elastin methacrylamide, cellulose acrylate, cellulose methacrylamide, dextran methacrylate, dextran methacrylamide, heparin methacrylate, heparin methacrylamide, N-isopropyl acrylamide (NIPAAm), chitosan methacrylate, chitosan methacrylamide, polyethylene glycol norbornene, polyethylene glycol dithiol, thiolated gelatin, thiolated chitosan, thiolated hyaluronic acid, thiolated silk, PEG based peptide conjugates, decellularized ECM of any tissue/organ, plastic, metal, including gallium or indium, or alloys, including Field's metal, gallium-indium, gallium-tin, and gallium-indium-tin, supercooled liquid metal, or any combination thereof.

Embodiment 30. The method of any one of Embodiments 20-29, wherein the cast structure is removed by dissolution or degradation, by one or more chemical processes under acidic or basic conditions, collagenase incubation with a protease or peptidase, including trypsin, collagenase, proteinase k, cathepsin K, or any combination thereof, or by one or more physical processes via a mechanical process, swelling, drying, heating the cast structure to liquify the first material, or a light process due to presence of photolabile linkers in the initial structure, or any combination thereof.

Embodiment 31. The method of any one of Embodiments 20-30, wherein the initial structure comprises a vascular topology within a tissue or organ.

Embodiment 32. The method of any one of Embodiments 20-31, wherein the final structure is an artificial tissue or organ.

Embodiment 33. The method of any one of Embodiments 20-32, further comprising: modifying a surface of the cast structure prior to casting the second material.

Embodiment 34. The method of Embodiment 33, wherein modifying the surface of the cast structure further comprises roughening or smoothing of the surface, coating with a layer of collagen, coating a portion of the surface with collagen, electroplating, performing electrolysis, using ferro fluid in magnetic field to perform a surface treatment of the surface, or any combination thereof.

Embodiment 35. The method of any one of Embodiments 20-34, further comprising: forming one or more coatings on the cast structure prior to casting the second material.

Embodiment 36. The method of any preceding Embodiment 35, wherein the one or more coatings comprises a biocompatible material, a hemocompatible material, cells, a cell-adhesive material, collagen, gelatin, fibronectin, laminin, polylysine, PEG based peptide conjugates, polyurethane, or any combination thereof, or a metal comprising copper, nickel, or gold.

Embodiment 37. The method of any one of Embodiments 20-36, wherein the one or more coatings has a thickness between 10 nm and 1000 μm.

Embodiment 38. The method of any one of Embodiments 20-37, wherein the one or more coatings comprises a multilayer coating, wherein the multilayer coating is obtained by forming a coating at least two times.

Embodiment 39. The method of Embodiment 38, wherein the multilayer coating on the cast structure comprises at least two different coating materials.

Embodiment 40. A method of preparing a structure, comprising: providing an initial structure; casting a first material in one or more void volumes of the initial structure; removing the initial structure from the first material; obtaining a cast structure comprising the first material; coating a second material on the cast structure; removing the first material from the coated second material; and obtaining a final structure comprising the coated second material.

Embodiment 41. The method of Embodiment 40, wherein the initial structure is a hydrogel matrix structure generated by additive or subtractive manufacturing.

Embodiment 42. The method of any one of Embodiments 40 or 41, wherein the initial structure comprises a wax, a plastic, or polyvinyl alcohol, polyethylene glycol diacrylate (PEGDA) having 250-35,000 Da, PEG-norbornene, MMP-sensitive PEGs (PEG-MMP), gelatin methacrylate, or any combination thereof.

Embodiment 43. The method of any one of Embodiments 40-42, wherein the one or more void volumes forms a vascular topology.

Embodiment 44. The method of any one of Embodiments 40-43, wherein the initial structure is post-processed via washing or equilibrating in a solvent, or by crosslinking in a lightbox.

Embodiment 45. The method of any one of Embodiments 40-44, wherein the first material comprises a thermoreversible material, metal or liquid metal including gallium, alloys including Field's metal, gallium-indium, gallium-tin, and gallium-indium-tin, carbohydrate glass, pluronic, low molecular weight PEG, PCL, gelatin, wax, or any combination thereof.

Embodiment 46. The method of any one of Embodiments 40-45, wherein casting the first material in the one or more void volumes of the initial structure comprises: filling the first material in the one or more void volumes of the initial structure at a first temperature; and solidifying the first material at a second temperature, wherein the first material at the first temperature and the first material at the second temperature have different physical states.

Embodiment 47. The method of any one of Embodiments 40-46, wherein the initial structure is removed by dissolution or degradation, by one or more chemical processes under acidic or basic conditions, collagenase incubation with a protease or peptidase, including trypsin, collagenase, proteinase k, cathepsin K, or any combination thereof, or by one or more physical processes via a mechanical process, swelling, drying, heating, or via a light process due to presence of photolabile linkers in the initial structure, or any combination thereof.

Embodiment 48. The method of any one of Embodiments 40-47, wherein the second material comprises a biocompatible material, a hemocompatible material, cells, a cell-adhesive material, collagen, gelatin, fibronectin, laminin, polylysine, PEG based peptide conjugates, polyurethane, or any combination thereof, or a metal comprising copper, nickel, or gold.

Embodiment 49. The method of any one of Embodiments 40-48, wherein the second material is coated via dip-coating, spray coating, powder coating, vapor deposition, electroplating, or via oxidation and/or reduction reactions, or a combination thereof.

Embodiment 50. The method of any one of Embodiments 40-49, wherein the second material is coated to a thickness between 10 nm and 1000 μm.

Embodiment 51. The method of any one of Embodiments 40-50, wherein the second material is coated at least two times to form a multilayer coating.

Embodiment 52. The method of Embodiment 51, wherein the multilayer coating on the cast structure comprises at least two different coating materials.

Embodiment 53. The method of any one of Embodiments 40-52, wherein the first material is removed by dissolution or degradation, by one or more chemical processes under acidic or basic conditions, collagenase incubation with a protease or peptidase, including trypsin, collagenase, proteinase k, cathepsin K, or any combination thereof, or by one or more physical processes via a mechanical process, swelling, drying, heating the cast structure to liquify the first material, or a light process due to presence of photolabile linkers in the initial structure, or any combination thereof.

Embodiment 54. The method of any one of Embodiments 40-53, wherein the initial structure comprises a vascular topology within a tissue or organ.

Embodiment 55. The method of any one of Embodiments 40-54, wherein the final structure is an artificial tissue or organ.

Embodiment 56. The method of any one of Embodiments 40-55, further comprising: modifying a surface of the cast structure.

Embodiment 57. The method of Embodiment 56, wherein modifying the surface of the cast structure further comprises roughening or smoothing of the surface, coating with a layer of collagen, coating a portion of the surface with collagen, electroplating, performing electrolysis, using ferro fluid in magnetic field to perform a surface treatment of the surface, or any combination thereof.

Embodiment 58. A method of preparing a structure, comprising: providing an initial structure; casting a first material in one or more void volumes of the initial structure; removing the initial structure from the first material; obtaining a cast structure comprising the first material; coating a second material on the cast structure; casting a third material using the coated cast structure; removing the first material; and obtaining a final structure.

Embodiment 59. The method of Embodiment 58, wherein the initial structure is a hydrogel matrix structure generated by additive or subtractive manufacturing.

Embodiment 60. The method of any one of Embodiments 58 or 59, wherein the initial structure comprises a wax, a plastic, or polyvinyl alcohol, polyethylene glycol diacrylate (PEGDA) having 250-35,000 Da, PEG-norbornene, MMP-sensitive PEGs (PEG-MMP), gelatin methacrylate, or any combination thereof.

Embodiment 61. The method of any one of Embodiments 58-60, wherein the one or more void volumes forms a vascular topology.

Embodiment 62. The method of any one of Embodiments 58-61, wherein the initial structure is post-processed via washing or equilibrating in a solvent, or by crosslinking in a lightbox.

Embodiment 63. The method of any one of Embodiments 58-62, wherein the first material comprises a thermoreversible material, metal or liquid metal including gallium, alloys including Field's metal, gallium-indium, gallium-tin, and gallium-indium-tin, carbohydrate glass, pluronic, low molecular weight PEG, PCL, gelatin, wax, or any combination thereof.

Embodiment 64. The method of any one of Embodiments 58-63, wherein casting the first material in the one or more void volumes of the initial structure comprises: filling the first material in the one or more void volumes of the initial structure at a first temperature; and solidifying the first material at a second temperature, wherein the first material at the first temperature and the first material at the second temperature have different physical states.

Embodiment 65. The method of any one of Embodiments 58-64, wherein the initial structure is removed by dissolution or degradation, by one or more chemical processes under acidic or basic conditions, collagenase incubation with a protease or peptidase, including trypsin, collagenase, proteinase k, cathepsin K, or any combination thereof, or by one or more physical processes via a mechanical process, swelling, drying, heating, or via a light process due to presence of photolabile linkers in the initial structure, or any combination thereof.

Embodiment 66. The method of any one of Embodiments 58-65, wherein the second material comprises a biocompatible material, a hemocompatible material, cells, a cell-adhesive material, collagen, gelatin, fibronectin, laminin, polylysine, PEG based peptide conjugates, polyurethane, or any combination thereof, or a metal comprising copper, nickel, or gold.

Embodiment 67. The method of any one of Embodiments 58-66, wherein the second material is coated via dip-coating, spray coating, powder coating, vapor deposition, electroplating, or via oxidation and/or reduction reactions, or a combination thereof.

Embodiment 68. The method of any one of Embodiments 58-67, wherein the second material is coated to a thickness between 10 nm and 1000 μm.

Embodiment 69. The method of any one of Embodiments 58-68, wherein the second material is coated at least two times to form a multilayer coating.

Embodiment 70. The method of any one of Embodiments 58-69, wherein the multilayer coating on the cast structure comprises at least two different coating materials.

Embodiment 71. The method of any one of Embodiments 58-70, wherein the third material comprises a biomaterial comprising silk, collagen, gelatin, fibrin, synthetic peptides, hyaluronic acid, polymers comprising alginate, polyurethane, polycaprolactone (PCL), elastomers, collagen methacrylate, collagen methacrylamide, gelatin methacrylate, gelatin methacrylamide, silk methacrylate, silk methacrylamide, hyaluronic acid methacrylate, hyaluronic acid methacrylamide, pluronic diacrylate, pluronic methacrylamide, chondroitin sulfate methacrylate, chondroitin sulfate methacrylamide, elastin methacrylate, elastin methacrylamide, cellulose acrylate, cellulose methacrylamide, dextran methacrylate, dextran methacrylamide, heparin methacrylate, heparin methacrylamide, N-isopropyl acrylamide (NIPAAm), chitosan methacrylate, chitosan methacrylamide, polyethylene glycol norbornene, polyethylene glycol dithiol, thiolated gelatin, thiolated chitosan, thiolated hyaluronic acid, thiolated silk, PEG based peptide conjugates, decellularized ECM of any tissue/organ, plastic, metal, including gallium or indium, or alloys, including Field's metal, gallium-indium, gallium-tin, and gallium-indium-tin, supercooled liquid metal, or any combination thereof.

Embodiment 72. The method of any one of Embodiments 58-71, wherein the first material is removed by dissolution or degradation, by one or more chemical processes under acidic or basic conditions, collagenase incubation with a protease or peptidase, including trypsin, collagenase, proteinase k, cathepsin K, or any combination thereof, or by one or more physical processes via a mechanical process, swelling, drying, heating the cast structure to liquify the first material, or a light process due to presence of photolabile linkers in the initial structure, or any combination thereof.

Embodiment 73. The method of any one of Embodiments 58-72, wherein the initial structure comprises a vascular topology within a tissue or organ.

Embodiment 74. The method of any one of Embodiments 58-73, wherein the final structure is an artificial tissue or organ.

Embodiment 75. The method of any one of Embodiments 58-74, further comprising: modifying a surface of the cast structure prior to coating the second material.

Embodiment 76. The method of Embodiment 75, wherein modifying the surface of the cast structure further comprises roughening or smoothing of the surface, coating with a layer of collagen, coating a portion of the surface with collagen, electroplating, performing electrolysis, using ferro fluid in magnetic field to perform a surface treatment of the surface, or any combination thereof.

Embodiment 77. A method of preparing a structure, comprising: providing a first initial structure and a second initial structure; casting a first material in one or more first void volumes of the first initial structure and in one or more second void volumes of the second initial structure; removing the first initial structure and the second initial structure; obtaining a first cast structure and a second cast structure each comprising the first material; assembling the first cast structure and the second cast structure; obtaining an assembled structure comprising the first cast structure and the second cast structure; casting a third material using the assembled structure; removing the first material; and obtaining a final structure.

Embodiment 78. The method of Embodiment 77, wherein at least one of the first initial structure or the second initial structure is a hydrogel matrix structure generated by additive or subtractive manufacturing.

Embodiment 79. The method of any one of Embodiments 77 or 78, wherein at least one of the first initial structure or the second initial structure comprises a wax, a plastic, or polyvinyl alcohol, polyethylene glycol diacrylate (PEGDA) having 250-35,000 Da, PEG-norbornene, MMP-sensitive PEGs (PEG-MMP), gelatin methacrylate, or any combination thereof.

Embodiment 80. The method of any one of Embodiments 77-79, wherein the one or more first void volumes forms a first vascular topology and the one or more second void volumes forms a second vascular topology.

Embodiment 81. The method of any one of Embodiments 77-80, wherein at least one of the first initial structure or the second initial structure is post-processed via washing or equilibrating in a solvent, or by crosslinking in a lightbox.

Embodiment 82. The method of any one of Embodiments 77-81, wherein the first material comprises a thermoreversible material, metal or liquid metal including gallium, alloys including Field's metal, gallium-indium, gallium-tin, and gallium-indium-tin, carbohydrate glass, pluronic, low molecular weight PEG, PCL, gelatin, wax, or any combination thereof.

Embodiment 83. The method of any one of Embodiments 77-82, wherein casting the first material in the one or more first void volumes and in the one or more second void volumes comprises: filling the first material at a first temperature; and solidifying the first material at a second temperature, wherein the first material at the first temperature and the first material at the second temperature have different physical states.

Embodiment 84. The method of any one of Embodiments 77-83, wherein at least one of the first initial structure or the second initial structure is removed by dissolution or degradation, by one or more chemical processes under acidic or basic conditions, collagenase incubation with a protease or peptidase, including trypsin, collagenase, proteinase k, cathepsin K, or any combination thereof, or by one or more physical processes via a mechanical process, swelling, drying, heating, or via a light process due to presence of photolabile linkers in the initial structure, or any combination thereof.

Embodiment 85. The method of any one of Embodiments 77-84, further comprising: modifying a surface of at least one of the first cast structure or the second cast structure.

Embodiment 86. The method of any one of Embodiments 77-85, further comprising: coating one or more second materials on the first cast structure and the second cast structure.

Embodiment 87. The method of Embodiment 86, wherein the one or more second materials comprises a biocompatible material, a hemocompatible material, cells, a cell-adhesive material, collagen, gelatin, fibronectin, laminin, polylysine, PEG based peptide conjugates, polyurethane, or any combination thereof, or a metal comprising copper, nickel, or gold.

Embodiment 88. The method of any one of Embodiments 77-87, wherein the one or more second materials is coated on at least one of the first cast structure or the second cast structure via dip-coating, spray coating, powder coating, vapor deposition, electroplating, or via oxidation and/or reduction reactions, or a combination thereof.

Embodiment 89. The method of any one of Embodiments 77-88, wherein the one or more second materials is coated on at least one of the first cast structure or the second cast structure to a thickness between 10 nm and 1000 μm.

Embodiment 90. The method of any one of Embodiments 77-89, wherein the one or more second materials is coated on at least one of the first cast structure or the second cast structure at least two times to form a multilayer coating.

Embodiment 91. The method of Embodiment 90, wherein the multilayer coating on the at least one of the first cast structure or the second cast structure comprises at least two different coating materials.

Embodiment 92. The method of any one of Embodiments 77-91, wherein the one or more second materials that is coated on the first cast structure has a different thickness than the second material that is coated on the second cast structure.

Embodiment 93. The method of any one of Embodiments 77-92, wherein the one or more second materials that is coated on the first cast structure has a different surface roughness value than the second material that is coated on the second cast structure.

Embodiment 94. The method of any one of Embodiments 77-93, wherein the one or more second materials that is coated on the first cast structure is different from the one or more second materials that is coated on the second cast structure.

Embodiment 95. The method of any one of Embodiments 77-94, wherein nickel is coated on the first cast structure and gold is coated on the second cast structure.

Embodiment 96. The method of any one of Embodiments 77-95, wherein the assembled structure is a capacitor.

Embodiment 97. The method of any one of Embodiments 77-96, wherein at least one of the first cast structure or the second cast structure is removed by dissolution or degradation, by one or more chemical processes under acidic or basic conditions, collagenase incubation with a protease or peptidase, including trypsin, collagenase, proteinase k, cathepsin K, or any combination thereof, or by one or more physical processes via a mechanical process, swelling, drying, heating the first cast structure or the second cast structure to liquify the first material, or a light process due to presence of photolabile linkers in the initial structure, or any combination thereof.

Embodiment 98. The method of any one of Embodiments 77-97, wherein the first cast structure or the second cast structure is removed by heating to liquify one of the first cast structure or the second cast structure.

Embodiment 99. The method of any one of Embodiments 77-98, wherein modifying the surface comprises roughening or smoothing of the surface, coating with a layer of collagen, coating a portion of the surface with collagen, electroplating, performing electrolysis, using ferro fluid in magnetic field to perform a surface treatment of the surface, or any combination thereof.

Embodiment 100. The method of any one of Embodiments 77-99, wherein the third material comprises a biomaterial comprising silk, collagen, gelatin, fibrin, synthetic peptides, hyaluronic acid, polymers comprising alginate, polyurethane, polycaprolactone (PCL), elastomers, collagen methacrylate, collagen methacrylamide, gelatin methacrylate, gelatin methacrylamide, silk methacrylate, silk methacrylamide, hyaluronic acid methacrylate, hyaluronic acid methacrylamide, pluronic diacrylate, pluronic methacrylamide, chondroitin sulfate methacrylate, chondroitin sulfate methacrylamide, elastin methacrylate, elastin methacrylamide, cellulose acrylate, cellulose methacrylamide, dextran methacrylate, dextran methacrylamide, heparin methacrylate, heparin methacrylamide, N-isopropyl acrylamide (NIPAAm), chitosan methacrylate, chitosan methacrylamide, polyethylene glycol norbornene, polyethylene glycol dithiol, thiolated gelatin, thiolated chitosan, thiolated hyaluronic acid, thiolated silk, PEG based peptide conjugates, decellularized ECM of any tissue/organ, plastic, metal, including gallium or indium, or alloys, including Field's metal, gallium-indium, gallium-tin, and gallium-indium-tin, supercooled liquid metal, or any combination thereof.

Embodiment 101. The method of any one of Embodiments 77-100, wherein the assembled structure comprises one or more first features of the first cast structure and one or more second features of the second cast structure.

Embodiment 102. The method of any preceding Embodiment 101, further comprising: heating to expand the one or more first features or the one or more second features prior to casting the third material using the assembled structure.

Embodiment 103. The method of any one of Embodiments 77-102, further comprising: cooling to shrink the one or more first features or the one or more second features prior to casting the third material using the assembled structure.

Embodiment 104. The method of any one of Embodiments 77-103, wherein the one or more first features and the one or more second features are separated by a distance of larger than 1 μm at a nearest point of separation between the one or more first features and the one or more second features.

Embodiment 105. The method of any one of Embodiments 77-104, wherein the final structure comprises one or more final first features that is a negative mold of the one or more first features of the first cast structure and one or more final second features that is a negative mold of the one or more second features of the second cast structure.

Embodiment 106. The method of any one of Embodiments 77-105, wherein the one or more final first features and the one or more final second features are separated by a distance of less than 10 μm at a nearest point of separation between the one or more final first features and the one or more final second features.

Embodiment 107. The method of any one of Embodiments 77-106, wherein at least one of the first initial structure or the second initial structure comprises a vascular topology within a tissue or organ.

Embodiment 108. The method of any one of Embodiments 77-107, wherein the final structure is an artificial tissue or organ.

Embodiment 109. A structure produced according to any preceding Embodiment.

Embodiment 110. The structure of Embodiment 109, comprising: a fluid channel for casting one or more of the first and second materials.

Embodiment 111. The structure of any preceding Embodiment, wherein the fluid channel is within a void volume of the initial structure.

Embodiment 112. The structure of any preceding Embodiment, wherein the fluid channel comprises one or more constrictions to regulate a flow of injectable material.

Embodiment 113. The structure of Embodiment 112, wherein the one or more constrictions is designed as a terminal feature.

Embodiment 114. The structure of any preceding Embodiment, wherein the one or more constrictions is configured as a valve to terminate a void volume after filling the injectable material.

Embodiment 115. A system for producing a structure according to any of preceding Embodiment, the system comprising an initial structure comprising a hydrogel, a chamber to house the hydrogel, a manifold to connect to hydrogel vessel inlets and outlets, one or more tubing connected to the manifold, one or more pumps, one or more fluid reservoirs, and/or one or more waste containers.

Embodiment 116. The system of Embodiment 115, wherein the one or more pumps comprises a syringe pump or a peristaltic pump.

Embodiment 117. The system of any preceding Embodiment, wherein the one or more pumps are used to fill an injectable material into one or more voids within the initial structure.

Embodiment 118. The system of any preceding Embodiment, wherein the one or more voids within the initial structure are filled using a positive or negative pressure.

Embodiment 119. The system of Embodiment 118, wherein an electrical gradient is applied to fill the one or more voids within the initial structure.

Embodiment 120. The system of any preceding Embodiment, wherein a vacuum is applied to fill the one or more voids within the initial structure.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

References to “or” may be construed as inclusive so that any terms described using “or” may indicate any of a single, more than one, and all of the described terms. The labels “first,” “second,” “third,” and so forth are not necessarily meant to indicate an ordering and are generally used merely to distinguish between like or similar items or elements.

Various modifications to the implementations described in this disclosure may be readily apparent to those skilled in the art, and the generic principles defined herein may be applied to other implementations without departing from the spirit or scope of this disclosure.

Thus, the claims are not intended to be limited to the implementations shown herein, but are to be accorded the widest scope consistent with this disclosure, the principles and the novel features disclosed herein.

What is claimed is:

1. A method of preparing a structure, comprising:
providing an initial structure;
casting a first material in one or more void volumes of the initial structure;
removing the initial structure from the first material;
obtaining a cast structure comprising the first material;
casting a second material using the cast structure;
removing the cast structure from the second material; and
obtaining a final structure comprising the second material.

2. The method of claim 1, wherein the initial structure is a hydrogel matrix structure generated by additive or subtractive manufacturing.

3. The method of claim 1, wherein the initial structure comprises a wax, a plastic, or polyvinyl alcohol, polyethylene glycol diacrylate (PEGDA) having 250-35,000 Da, PEG-norbornene, MMP-sensitive PEGs (PEG-MMP), gelatin methacrylate, or any combination thereof.

4. The method of claim 1, wherein the one or more void volumes forms a vascular topology.

5. The method of claim 1, wherein the initial structure is post-processed via washing or equilibrating in a solvent, or by crosslinking in a lightbox.

6. The method of claim 1, wherein the first material comprises a thermoreversible material, metal or liquid metal including gallium, alloys including Field's metal, gallium-indium, gallium-tin, and gallium-indium-tin, supercooled liquid metal, carbohydrate glass, pluronic, low molecular weight PEG, PCL, gelatin, wax, or any combination thereof.

7. The method of claim 1, wherein casting the first material in the one or more void volumes of the initial structure comprises:
filling the first material in the one or more void volumes of the initial structure at a first temperature; and
solidifying the first material at a second temperature, wherein the first material at the first temperature and the first material at the second temperature have different physical states.

8. The method of claim 1, wherein casting the first material in the one or more void volumes of the initial structure comprises:
injecting the first material in the one or more void volumes of the initial structure at a first temperature;
supercooling the first material; and
crystalizing the first material at a second temperature.

9. The method of claim 1, wherein the initial structure is removed by dissolution or degradation, by one or more chemical processes under acidic or basic conditions, collagenase incubation with a protease or peptidase, including trypsin, collagenase, proteinase k, cathepsin K, or any combination thereof, or by one or more physical processes via a mechanical process, swelling, drying, heating, or via a light process due to presence of photolabile linkers in the initial structure, or any combination thereof.

10. The method of claim 1, wherein the second material comprises a biomaterial comprising silk, collagen, gelatin, fibrin, synthetic peptides, hyaluronic acid, polymers comprising alginate, polyurethane, polycaprolactone (PCL), elastomers, collagen methacrylate, collagen methacrylamide, gelatin methacrylate, gelatin methacrylamide, silk methacrylate, silk methacrylamide, hyaluronic acid methacrylate, hyaluronic acid methacrylamide, pluronic diacrylate, pluronic methacrylamide, chondroitin sulfate methacrylate, chondroitin sulfate methacrylamide, elastin methacrylate, elastin methacrylamide, cellulose acrylate, cellulose methacrylamide, dextran methacrylate, dextran methacrylamide, heparin methacrylate, heparin methacrylamide, N-isopropyl acrylamide (NIPAAm), chitosan methacrylate, chitosan methacrylamide, polyethylene glycol norbornene, polyethylene glycol dithiol, thiolated gelatin, thiolated chitosan, thiolated hyaluronic acid, thiolated silk, PEG based peptide conjugates, decellularized ECM of any tissue/organ, plastic, metal, including gallium or indium, or alloys, including Field's metal, gallium-indium, gallium-tin, and gallium-indium-tin, supercooled liquid metal, or any combination thereof.

11. The method of claim 1, wherein the cast structure is removed by dissolution or degradation, by one or more chemical processes under acidic or basic conditions, collagenase incubation with a protease or peptidase, including trypsin, collagenase, proteinase k, cathepsin K, or any combination thereof, or by one or more physical processes via a mechanical process, swelling, drying, heating the cast structure to liquify the first material, or a light process due to presence of photolabile linkers in the initial structure, or any combination thereof.

12. The method of claim 1, wherein the initial structure comprises a vascular topology within a tissue or organ.

13. The method of claim 1, wherein the final structure is an artificial tissue or organ.

14. The method of claim 1, further comprising:
modifying a surface of the cast structure prior to casting the second material.

15. The method of claim 14, wherein modifying the surface of the cast structure further comprises roughening or smoothing of the surface, coating with a layer of collagen, coating a portion of the surface with collagen, electroplating, performing electrolysis, using ferro fluid in magnetic field to perform a surface treatment of the surface, or any combination thereof.

16. The method of claim 1, further comprising:
forming one or more coatings on the cast structure prior to casting the second material.

17. The method of claim 16, wherein the one or more coatings comprises a biocompatible material, a hemocompatible material, cells, a cell-adhesive material, collagen, gelatin, fibronectin, laminin, polylysine, PEG based peptide conjugates, polyurethane, or any combination thereof, or a metal comprising copper, nickel, or gold.

18. The method of claim 16, wherein the one or more coatings has a thickness between 10 nm and 1000 μm.

19. The method of claim 16, wherein the one or more coatings comprises a multilayer coating, wherein the multilayer coating is obtained by forming a coating at least two times.

20. The method of claim 19, wherein the multilayer coating on the cast structure comprises at least two different coating materials.
